(19) United States
(12) Patent Application Publication
(10) Pub. No.: US 2018/0303090 A1
(43) Pub. Date: Oct. 25, 2018

(54) TREATMENT COMPOSITIONS PROVIDING AN ANTIMICROBIAL BENEFIT

(71) Applicant: Reckitt Benckiser LLC, Parsippany, NJ (US)

(72) Inventors: Avinash BUDHIAN, Montvale, NJ (US); Sarah Frances DE SZALAY, Montvale, NJ (US); Richard GILES, Gothenburg (SE); Aleksandra KRUSZEWSKA, Montvale, NJ (US); Pamela MCGOWAN, Montvale, NJ (US)

(21) Appl. No.: 15/770,710

(22) PCT Filed: Oct. 17, 2016
(86) PCT No.: PCT/GB2016/053214

§ 371 Date: Apr. 24, 2018

Related U.S. Application Data
(60) Provisional application No. 62/248,354, filed on Oct. 30, 2015.

(51) Int. Cl. A01N 37/36; A61K 31/194; A61K 31/19; A01N 25/30; A61K 47/20; A61K 47/18; A61K 47/26

(52) U.S. Cl. CPC A01N 37/36; A61K 31/194; A61K 31/19; A61K 47/26; A61K 47/20; A61K 47/18; A01N 25/30

(57) ABSTRACT

Treatment compositions which may be use to impart an antimicrobial benefit to animate and inanimate surfaces, e.g, topical compositions and hard surface and soft surface treatment compositions comprising specific surfactant comprising anionic surfactants, or anionic and nonionic surfactant systems.

The present invention relates to treatment compositions. More particularly the present composition relates to treatment compositions which may be use to impart an antimicrobial benefit to animate and inanimate surfaces, e.g, topical compositions and hard surface and soft surface treatment compositions.

WO 2008/031104 discloses a bovine barrier teat dip compositions which necessarily comprise lactic acid with sodium octane sulfonate, and optionally further includes sodium lauryl sulfate, wherein the compositions were at pHs 4.00 or less which provided good antimicrobial benefits, according to the EN 1656 test protocol. The test results are attributed to a believed synergy of the lactic acid with the sodium octane sulfonate.

U.S. Pat. No. 8,268,334 demonstrate inantimate hard surface treatment compositions which necessarily includes an acid constituent which includes a ternary system of lactic acid, citric acid and malic acid, an organic solvent constituent, an anionic solvent constituent, and a noionic surfactant constituent, wherein the compositions are at pH of 3.5 or less.

WO 02/097020 discloses bactericidal liquid detergent compositions which include a surfactant selected from anionic, nonionic, cationic and amphoteric surfactants, an antimicrobial agent selected from the group consisting of benzoic acid, sorbic acid, trimethyl dodecanetriol, or dehydroxyacetic acid, or salts forms thereof, an aromatic sulfonate hydrotrope, a water soluble hydroxyl-containing solvent, and water. The actual antimicrobial effects of the compositions are not reported.

Although the prior art discloses a myriad of treatment compositions which provide some degree of antimicrobial benefit, some of which are specifically formulated for use on inanimate surfaces and other specifically formulated for use on dermal surfaces of a human or animal body, a large number of these rely upon the inclusion of synthetically produced organic compounds, e.g. Triclosan, in order to provide an antimicrobial benefit. Such is however not always desirable from a consumer acceptance and/or toxicological standpoint. Other known art antimicrobial compounds, including antimicrobially effective quaternary ammonium compounds such as alkylbenzyl dimethyl quaternary ammonium chlorides and alkylated quaternary ammonium chlorides are highly effective but if used in excess may induce dermal irritation. Furthermore many of such known art compositions are frequently directed to be applied, and after a short period of time (e.g, 30 seconds-5 minutes) are rinsed with water from a treated surface, which dilutes or removes the synthetically produced organic compounds from the surface upon which they have been applied. Thus, a number of shortcomings are still known to regarding to such compositions, and it is to these and further shortcomings which the present invention is directed.

In a first aspect of the present invention there is provided an acidic aqueous treatment composition having a pH not in excess of 4.5 which necessarily comprises:

as an antimicrobial constituent, lactic acid, citric acid, substituted lactic acid, substituted citric acid, tartaric acid, substituted tartaric acid, derivatives thereof and/or salts thereof; and,

an antimicrobially enhancing effective amount of a ternary anionic surfactant system which necessarily comprises one or more of each of: (a) a secondary alkane sulfonate surfactant compound(s), (b) an N-acyl sarcosinate compound and (c) an anionic hydrotrope surfactant compound(s), preferably a cumene sulfonate compound, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae. Further preferably the ternary anionic surfactant system boosts the antimicrobial efficacy of the antimicrobial constituent present as compared to like compositions wherein (a), (b) and (c) are omitted.

In a second aspect the present invention provides an acidic aqueous treatment composition having a pH not in excess of 4.5 which treatment composition necessarily comprises:

as an antimicrobial constituent, lactic acid, citric acid, substituted lactic acid, substituted citric acid, tartaric acid, substituted tartaric acid, derivatives thereof and/or salts thereof; and,

an antimicrobially enhancing effective amount of a binary anionic surfactant system which necessarily comprises one or more of each of: (a) a secondary alkane sulfonate surfactant compound(s), and (c) an anionic hydrotrope surfactant compound(s) which is preferably selected from a xylene sulfonate compound and a cumene sulfonate compound, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, Preferably the binary anionic surfactant system boosts the antimicrobial efficacy of the antimicrobial constituent present as compared to like compositions wherein (a) and (c) are omitted.

In a third aspect, the present invention provides an acidic aqueous treatment composition having a pH not in excess of 4.5, which composition is particularly adapted for the treatment of hard and/or soft surfaces which have been contacted by a non-human animal, e.g. canines, felines, reptiles which are typically considered household pets, which treatment composition necessarily comprises:

as an antimicrobial constituent, lactic acid, citric acid, substituted lactic acid, substituted citric acid, tartaric acid, substituted tartaric acid, derivatives thereof and/or salts thereof; and,
a ternary surfactant constituent system which necessarily comprises one or more of each of: (a) one or more alkyl sulfonate surfactant compound(s) selected from alkylbenzene sulfonate compound(s) and a secondary alkane sulfonate surfactant compound(s); (b) one or more alkylglycoside nonionic surfactant compound(s); and; (c) an aromatic hydrotrope compound(s), especially preferably a cumene sulfonate compound, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, Preferably the ternary surfactant constituent system boosts the antimicrobial efficacy of the antimicrobial constituent present as compared to a like composition wherein (a), (b) and (c) are omitted;

According to a variant of the third aspect of the invention there is provided an acidic aqueous treatment composition having a pH not in excess of 4.5, which composition is particularly adapted for the treatment of hard and/or soft surfaces which have been contacted by a non-human animal, e.g. canines, felines, reptiles which are typically considered household pets, which necessarily treatment composition comprises at least:

as an antimicrobial constituent, one or more organic acids selected from lactic acid, citric acid, substituted lactic acid, substituted citric acid, tartaric acid, substituted tartaric acid, derivatives thereof and/or salts thereof; and,

a quaternary surfactant constituent system which necessarily comprises one or more of each of: (a) an alkyl sulfonate surfactant compound(s) selected from an alkylbenzene sulfonate compound and a secondary alkane sulfonate surfactant compound(s); (b) one or more alkylglycoside nonionic surfactant compound(s); (c) an aromatic hydrotrope compound(s), especially preferably a cumene sulfonate compound; and, (d) an N-acyl sarcosinate compound(s), and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae. Preferably the quaternary constituent system boosts the antimicrobial efficacy of the disinfecting constituent present as compared to a like composition wherein (a), (b), (c) and (d) are omitted.

In a fourth aspect the present invention provides an acidic, largely aqueous hard surface treatment composition having a pH not in excess of 4.5 which features low toxicity to humans and animals, and which also provides a good antimicrobial effect against undesired microorganisms, which is optionally but preferably provided with a carrier substrate, which hard surface treatment composition necessarily comprises:

lactic acid, citric acid, substituted lactic acid, substituted citric acid, tartaric acid, substituted tartaric acid, derivatives thereof and/or salts thereof; and,

a binary system of anionic compounds which necessarily includes: (a) one or more alkylbenzene sulfonate surfactant compounds; and (b) an anionic aromatic hydrotrope compound, especially preferably which anionic hydrotrope includes an aryl moiety and is particularly preferably selected from the group consisting of: cumene sulfonate and/or xylene sulfonate or salts thereof, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae. Preferably the binary system boosts the antimicrobial efficacy of the disinfecting constituent present as compared to a like composition wherein (a) and (b) are omitted; and further wherein the treatment composition preferably exhibits a pH of from about 1 to about 4.5, and particularly preferably from about 1.8 to about 3.05.

A first variant of the foregoing fourth recited aspect provides an acidic, largely aqueous hard surface treatment composition having a pH not in excess of 4.5 which features low toxicity to humans and animals, but which provides a good antimicrobial effect against undesired microorganisms, which is optionally but preferably supplied with a carrier substrate, which hard surface treatment composition necessarily comprises:

as an antimicrobial constituent, lactic acid, citric acid, substituted lactic acid, substituted citric acid, tartaric acid, substituted tartaric acid, derivatives thereof and/or salts thereof; and,

a ternary system of surfactant compounds which necessarily includes: (a) one or more alkylbenzene sulfonate surfactant compounds; and (b) one or more anionic aromatic hydrotrope compounds, especially preferably which anionic hydrotrope includes an aryl moiety and is particularly preferably selected from the group consisting of: cumene sulfonate and/or xylene sulfonate or salts thereof, and (c) one or more further anionic surfactant(s) other than (a) and (b) which further anionic surfactant is preferably at least one, but preferably at least two anionic surfactants selected from linear alkane sulfonate and/or a linear alkane sulfate, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae. Preferably the ternary system boosts the antimicrobial efficacy of the disinfecting constituent present as compared to a like composition wherein (a), (b) and (c) are omitted; and further preferably, wherein the treatment composition exhibits a pH of from about 1 to about 4.5, preferably from about 2 to about 3.05.

A second variant of the foregoing fourth recited aspect provides an acidic, largely aqueous hard surface treatment composition having a pH not in excess of 4.5 which features low toxicity to humans and animals, but which provides a good antimicrobial effect against undesired microorganisms, which is optionally but preferably supplied with a carrier substrate, which hard surface treatment composition necessarily comprises:

as an antimicrobial constituent, lactic acid, citric acid, substituted lactic acid, substituted citric acid, tartaric acid, substituted tartaric acid, derivatives thereof and/or salts thereof; and,

a quaternary system of surfactant compounds which necessarily includes: (a) one or more alkylbenzene sulfonate surfactant compounds; and (b) one or more anionic aromatic hydrotrope compounds, especially preferably which anionic hydrotrope includes an aryl moiety and is particularly preferably selected from the group consisting of: cumene sulfonate and/or xylene sulfonate or salts thereof, (c) one or more further anionic surfactant(s) other than (a) and (b) which further anionic surfactant is preferably at least one, but preferably at least two anionic surfactants selected from linear alkane sulfonate and/or a linear alkane sulfate, and (d) one or more alkylglucoside nonionic surfactant compounds, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae. Preferably the quaternary system boosts the antimicrobial efficacy of the disinfecting constituent present as compared to a like composition wherein (a), (b), (c) and (d) are omitted; and further preferably, the treatment composition exhibits a pH of from about 1 to about 4.5, preferably from about 2 to about 3.05.

Particularly preferred embodiments of the fourth aspect (and variants thereof) of the invention meet the requirements of 40 CFR § 180.940—Tolerance exemptions for active and inert ingredients for use in antimicrobial formulations (Food contact surface sanitizing solutions), viz., are considered “food safe”. Preferred embodiments of the fourth aspects of the invention do not require a subsequent aqueous rinse following treatment of a surface with compositions according to the fourth aspect, and variants thereof, of the invention.

According to a fifth aspect of the invention wherein are provided acidic compositions having a pH of about 4.5 or less and in which an antimicrobial constituent selected from salicylic acid and lactic acid may be omitted, but citric acid is present concurrently with a ternary anionic surfactant system is present and which system necessarily comprises one or more of each of: (a) a secondary alkane sulfonate surfactant compound(s), (b) an N-acyl sarcosinate compound(s) and (c) an anionic hydrotrope surfactant compound(s) is necessarily present and which (a), (b) and (c) provide an effective antimicrobial benefit against undesired microorganisms, preferably one or more of the bacteria selected from the genus: Salmonella, especially Salmonella enterica, and/or the genus Staphylococcus, especially Staphylococcus aureus.

Preferably the treatment compositions of the invention exhibit an antimicrobial effect (preferably sanitizing or disinfecting) and preferably a broad spectrum antimicrobial effect, as demonstrated according to one or more of the following known test protocols: (i) AOAC Official Method 961.02 Germicidal Spray Products as Disinfectants, including also when modified for use with towelettes according to EPA Series 810 guidelines (OCSPP 810.2200: Disinfectants for Use on Hard Surfaces—Efficacy Data Recommendations), (ii) AOAC Official Method 955.14, 955.15 (preferably against S. aureus and/or S. enterica); (iii) European Standard EN 1276:2009—Chemical Disinfectants and Antiseptics—Quantitative suspension test for the evaluation of bactericidal activity of chemical disinfectants used in the food, industrial, domestic and institutional areas; (iiii) European Standard Surface Test EN 13697:2001—Chemical disinfectants and antiseptics—Quantitative non-porous surface test for the evaluation of bactericidal and/or fungicidal activity of chemical disinfectants used in food, industrial, domestic and institutional areas; (v) ASTM E1153—Standard Test Method for Efficacy of Sanitizers Recommended for Inanimate Non-Food Contact Surfaces. Preferred treatment compositions exhibit at least a 3 log₁₀ reduction of one or more undesired microorganisms (pathogens), preferably (in order of increasing preference) a log₁₀ reduction of at least 3.25, 3.5, 3.75, 4, 4.25, 4.5, 4.75, 5, 5.1, 5.2, 5.25 or greater of at least one, preferably at least two or more of: Salmonella enterica, Staphylococcus aureus, Pseudomonas aeruginosa, Escherichia coli, Enterococcus hirae, Candida albicans and Aspergillis niger according to one or more art recognized protocols, particularly one or more of the test protocols described herein.

In accordance with a further inventive embodiment, the present invention provides a carrier substrate, e.g. a woven or non-woven wipe substrate, which contains a quantity of a hard surface treatment composition according to any of the other aspects of the invention described above or otherwise disclosed hereinafter.

According to a still further embodiment, the present invention provides a surface treatment composition and/or carrier substrate containing a hard surface treatment according to any prior described aspect, or as hereinafter recited, which further comprises one or more further constituents which may provide additional technical and/or aesthetic attributes to the compositions, e.g., viscosity, cleansing, rinsing, pH adjusting agents, storage stability, etc., which attributes are frequently desired in compositions and/or carrier substrates containing the treatment compositions.

According to a further embodiment, the present invention provides a method for the treatment of an animate and/or inanimate surface upon which is known to be present or suspected to be present one or more undesired microorganisms, preferably one or more of bacteria selected from the genus: Salmonella, especially S. enterica, and/or of the genus Staphylococcus, especially Staphylococcus aureus and/or of the genus Pseudomonas, especially P. aeruginosa, and/or of the genus Eserichia, especially E. coli, and/or of the genus Enterococcus, and especially E. hirae, and/or further microorganisms (yeasts, fungi) of the genus Candida, preferably C. albicans and/or of the genus Aspergillis, preferably A. niger, which method includes the step of: applying, (optionally repeatably applying,) an antimicrobially effective amount of the hard surface treatment composition herein described to surface in order to reduce the incidence of the aforesaid microorganisms (pathogens) associated with and/or upon the said surface.

In a further embodiment the present invention provides as a vendible article, a container which comprises a treatment composition as herein described.

The foregoing as well as further aspects of the invention are described more fully in the following.

### Antimicrobial Constituent:

When present, the antimicrobial constituent is one or more organic acids selected from lactic acid, citric acid, substituted lactic acid, substituted citric acid, tartaric acid, substituted tartaric acid, derivatives thereof and/or salts thereof.

Nonlimiting examples of lactic acid and/or derivatives thereof include: lactic acid, salts thereof such as metal salts (e.g., sodium) as well as alkyl lactates such as the reaction products of a C₈-C₂₀ fatty alcohol with lactic acid. Preferred alkyl lactates include those represented by the following general structural formula (Ia):

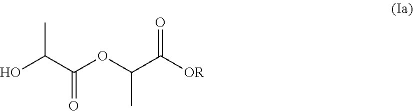

in which R is a C₈-C₂₀ alkyl moiety, preferably is a C₁₀-C₁₄ alkyl moiety and especially preferably is predominantly (at least 85%, more preferably at least 90%, particularly preferably at least 95% and most preferably at least about 98%) of a C₁₂ alkyl moiety. The alkyl moiety may be branched but is preferably substantially linear. Further preferred alkyl lactates also include those which may be represented by the following general structural formula (Ib):

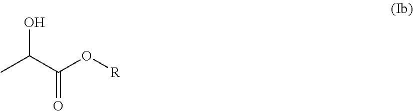

in which R is a C₈-C₁₈ alkyl moiety, preferably is a C₁₀-C₁₄ alkyl moiety and especially preferably is predominantly (at least 85%, more preferably at least 90%, particularly preferably at least 95% and most preferably at least about 98%) of a C₁₂ alkyl moiety. The alkyl moiety may be branched but is preferably substantially linear. Also useful are lactides as may be represented by the following formula (Ic):

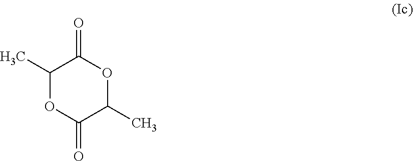

as well as polylactides as may be represented by the formula (Id):

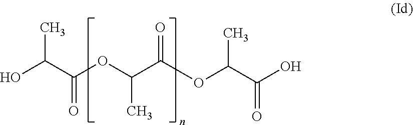

wherein n is an integer of at least 1, preferably n is an integer from 1-100 inclusive, and particularly preferably n is 1-3. Of course it is to be understood that other alkyl lactates not specifically encompassed by the compounds of formulae (Ia), (Ib), (Ic) and/or (Id) may also be utilized. The lactic acid and/or derivatives thereof may also be optionally substituted with one or more substituents, and by way of nonlimiting example, such substituents may be independently selected from alkyl, aryl, alcohol, ether, ester, cyanide, amide, amine, sulfate, phosphate, fluoro, chloro, bromo or iodo groups or carbonyl groups. The lactic acid, salt or derivative may also be provided deposited upon an inorganic carrier such as silica.

Nonlimiting examples of citric acid, substituted citric acid, derivatives thereof and salts thereof include citric acid in its free acid form as well as in the form of salts, and are preferably water soluble salts of citric acid. Nonlimiting examples of metal salts of citric acid include sodium, potassium and other metal salts of which the sodium salts are particularly preferred. Also useful are citric acid anhydrides and hydrates, particularly citric acid monohydrate.

Nonlimiting examples of tartaric acid, and derivatives thereof include but not limited to tartaric acid, tartrimides, tartrates, tartrate esters, tartaric acid esters, tartaric acid amides as well as salts of any of the foregoing, e.g, metal salts, inorganic salts. The tartaric acid may be any isomer thereof, e.g., L-tartaric acid, D-tartaric acid meso-tartaric acid. Non-limiting examples of tartaric acid derivatives include: dibenzyl L-tartrate, diisopropyl L-tartrate, diethyl L-tartrate, diethyl D-tartrate, dimethyl L-tartrate, dimethyl D-tartrate, dibenzoyl-L-tartaric acid monodimethylamide, dibenzoyl-D-tartaric acid monodimethylamide, L-tartaric acid mono-p-chloranilide, D-tartaric acid mono-p-chloranilide, dibenzoyl-D-tartaric monohydrate, dibenzoyl-L-tartaric monohydrate, di-p-toluoyl-D-tartaric acid, and di-p-toluoyl-L-tartaric acid. Of these, tartaric acid and/or salts thereof are preferred.

Generally, the antimicrobial constituent is present in an amount not in excess of about 5% wt. of the treatment composition of which it forms a part. Particularly preferred antimicrobial constituents according to a particular aspect (or variant of an aspect) of the invention, and the preferred amounts of the antimicrobial constituent are disclosed in one or more of the Examples.

### Secondary Alkane Sulfonate Surfactant Compounds:

In certain aspects of the invention a further essential constituent is one or more secondary alkane sulfonate surfactant compounds. Such include C₁₀-C₂₄, preferably C₁₄-C₁₇ olefin sulfonate compounds of the general formula (A):

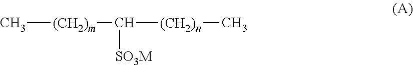

wherein m+n=an integer in the range of 7-21 inclusive, and is preferably an integer in the range of 11-14, inclusive, and M is a cation which is selected from an alkali metal such as sodium or potassioum, an alkaline earth metal such as calcium or magnesium, ammonium, or an alkanolamine such as monoalkanolamine (e.g. monoethanolamine), dialkanolamine (e.g. diethanolamine), trialkanolamine (e.g. triethanolamine). However M is preferably sodium.

The secondary alkane sulfonate compound(s) may be symmetrically branched or may be asymmetrically branched. What is meant by the term “symmetrically branched” is that the sulfonate moiety branches from a carbon atom which is at the midpoint between the two terminal carbons of the alkane portion of the molecule, while the term “asymmetrically branched” is that the sulfonate moiety branches from a carbon which is not equidistant from the two terminal carbon atoms. When present, the one or more secondary alkane sulfonate surfactant compounds do not exceed about 5% wt. of the inventive composition. The identity of and the preferred amounts of these compounds and preferred wt/wt ratios these compounds to other surfactant compounds are disclosed hereinafter in one or more of the Examples.

### Alkylbenzene Sulfonate Compounds:

One or more alkylbenzene sulfonate compounds are required constituents according to certain aspects of the invention. Non-limiting examples of useful alkylbenzene sulfonate compound(s) include those which may be represented by the following two structures:

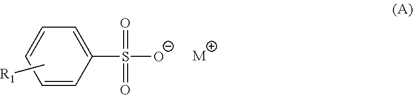

in which R₁ represent a linear C₈-C₂₄, preferably a C₁₀-C₁₄ alkyl or alkenyl moiety, and particularly preferably a C₁₆-C₁₈ alkyl moiety; and,

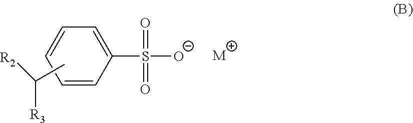

in which R₂ and R₃ independently represent a linear C₈-C₂₄, preferably a C₁₀-C₁₈ alkyl or alkenyl moiety, and particularly preferably a C₁₀-C₁₄ alkyl moiety; and preferably the total number of carbon atoms in the R₂ and R₃ moieties considered together is between 8-24 inclusive, preferably 10-18 inclusive, and most preferably 10-14 inclusive, and, in each (A) and (B), M is a counterion which renders the alkylbenzene sulfonate compound water soluble or water miscible, and is selected from an alkali metal such as sodium or potassium, an alkaline earth metal such as calcium or magnesium, ammonium, or an alkanolamine such as monoalkanolamine (e.g. monoethanolamine), dialkanolamine (e.g. diethanolamine), trialkanolamine (e.g. triethanolamine). However M is preferably sodium. When present, the one or more alkylbenzene sulfonate compounds do not exceed about 5% wt. of the inventive composition. The identity of and the preferred amounts of these compounds and preferred wt/wt ratios these compounds to other surfactant compounds are disclosed hereinafter in one or more of the Examples.

### Anionic Hydrotrope Surfactant Compounds:

Certain aspects of the invention require one or more anionic hydrotrope surfactant compounds, especially preferably which anionic hydrotrope includes an aryl moiety. Such compounds include benzene sulfonate, naphthalene sulfonate, cumene sulfonate, xylene sulfonate and toluene sulfonate, as well as derivatives thereof such as C₁-C₁₁ alkyl benzene sulfonates. The anionic hydrotrope compounds are often provided in a salt form with a suitable counterion, such as one or more alkali, or alkali earth metals, such as sodium, calcium or potassium, especially sodium. However, other water soluble cations such as ammonium, mono-, di- and tri-lower alkyl, i.e., C₁₋₄ alkanol ammonium groups can be used in the place of the alkali metal cations. Specific, albeit non-limiting examples of anionic hydrotrope compounds include alkyl benzene sulfonates such as isopropylbenzene sulfonate, xylene sulfonates such as 2,3-xylene sulfonates, 2,4-xylene sulfonates, and 4,6-xylene sulfonates, toluene sulfonates such as o-toluene sulfonates, m-toluene sulfonates, and p-toluene sulfonates, cumene sulfonates, as well as mixtures thereof. Preferred anionic hydrotrope compounds are provided as salts, preferably as sodium salts and/or potassium salts. Of the anionic hydrotrope compounds, particularly preferred are salts of anionic aromatic hydrotrope compounds which comprise a sulfonate moiety, including compounds selected from the group consisting of: benzene sulfonate, naphthalene sulfonate, cumene sulfonate, xylene sulfonate and toluene sulfonate, and particularly preferably, compounds selected from: cumene sulfonate and/or xylene sulfonate as well as salts thereof. When present, the one or more anionic hydrotrope surfactant compounds do not exceed about 5% wt. of the inventive composition. The identity of and the preferred amounts of these compounds and preferred wt/wt ratios these compounds to other surfactant compounds are disclosed hereinafter in one or more of the Examples.

### N-Acyl Sarcosinate Compounds:

According to certain aspects of the invention an essential constituent is a one or more N-acyl sarcosinate compounds. Such compounds are advantageously provided as metal salts, preferably an alkali metal salts, of an N-alkyl-N-acyl amino acids. These are salts derived from the reaction of N-alkyl substituted amino acids of the following formula:

R₁—NH—CH₂—COOH

where R₁ is a linear or branched chain lower alkyl of from 1 to 4 carbon atoms, especially a methyl, for example, aminoacetic acids such as N-methylaminoacetic acid (i.e. N-methyl glycine or sarcosine), N-ethyl-aminoacetic acid, N-butylaminoacetic acid, etc., with saturated natural or synthetic fatty acids having from 8 to 20 carbon atoms, especially from 10 to 14 carbon atoms, e.g. lauric acid, and the like.

The resultant reaction products are salts which may have the formula:

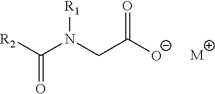

where M is an alkali metal ion such as sodium, potassium or lithium; R₁ is as defined above; and wherein R₂ represents a hydrocarbon chain, preferably a saturated hydrocarbon chain, having from about 6 to about 22 carbon atoms, and especially about 8 to about 18 carbon atoms.

Exemplary useful sarcosinate surfactants include cocoyl sarcosinate, lauroyl sarcosinate, myristoyl sarcosinate, palmitoyl sarcosinate, stearoyl sarcosinate and oleoyl sarcosinate, and tallow sarcosinate, of which preferred is the metal salts, preferably sodium salts, of lauroyl sarcosinate. When present, the one or more one or more N-acyl sarcosinate compounds do not exceed about 5% wt. of the inventive composition. The identity of and the preferred amounts of these compounds and preferred wt/wt ratios these compounds to other surfactant compounds are disclosed hereinafter in one or more of the Examples.

### Further Anionic Surfactant(s); Linear Alkane Sulfonate Compounds and Linear Alkane Sulfate Compounds:

Certain aspects of the invention also require that one or more linear alkane sulfonate compounds and/or linear alkane sulfate compounds be additionally present. Such include one or more of alcohol sulfates and sulfonates, alcohol phosphates and phosphonates, alkyl ester sulfates, alkyl diphenyl ether sulfonates, alkyl sulfates, alkyl ether sulfates, sulfate esters of an alkylphenoxy polyoxyethylene ethanol, alkyl monoglyceride sulfates, alkyl sulfonates, alkyl ether sulfates, alpha-olefin sulfonates, beta-alkoxy alkane sulfonates, alkyl ether sulfonates, ethoxylated alkyl sulfonates, alkyl monoglyceride sulfonates, alkyl carboxylates, alkyl ether carboxylates, alkyl alkoxy carboxylates having 1 to 5 moles of ethylene oxide, alkylpolyglycolethersulfates (containing up to 10 moles of ethylene oxide), sulfosuccinates, octoxynol or nonoxynol phosphates, taurates, fatty taurides, fatty acid amide polyoxyethylene sulfates, acyl glycerol sulfonates, fatty oleyl glycerol sulfates, alkyl phenol ethylene oxide ether sulfates, paraffin sulfonates, alkyl phosphates, isethionates, N-acyl taurates, alkyl succinamates and sulfosuccinates, alkylpolysaccharide sulfates, alkylpolyglucoside sulfates, alkyl polyethoxy carboxylates, sarcosinates and/or mixtures thereof. Such may be provided in their free acid forms or as salts, such as metal salts or other salt forms thereof which aid in the aqueous solubility or aqueous miscibility of the compound. Preferred are anionic surfactants selected from linear alkane sulfonate and/or a linear alkane sulfate which may be include water soluble salts or acids of the formula (ROSO₃)ₓM or (RSO₃)ₓM wherein R is preferably a C₆-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a mono-, di- or tri-valent cation, e. g., an alkali metal cation (e. g., sodium, potassium, lithium), or ammonium or substituted ammonium (e. g., methyl-, dimethyl-, and trimethyl ammonium cations and quaternary ammonium cations, such as tetramethyl-ammonium and dimethyl piperdinium cations and quaternary ammonium cations derived from alkylamines such as ethylamine, diethylamine, triethylamine, and mixtures thereof, and the like) and x is an integer, preferably 1 to 3, most preferably 1. Preferably also, at least one of each of alinear alkane sulfonate compounds a linear alkane sulfate compound is concurrently present. When present, the one or more linear alkane sulfonate compounds and/or linear alkane sulfate compounds do not exceed about 5% wt. of the inventive composition. The identity of and the preferred amounts of these compounds and preferred wt/wt ratios these compounds to other surfactant compounds are disclosed hereinafter in one or more of the Examples.

In certain preferred embodiments sodium octane sulfonates are excluded from the inventive compositions.

### Alkyl Glucoside Nonionic Surfactant Compounds:

Certain aspects of the invention require that one or more alkylglucoside (alkylglycoside) nonionic surfactant compounds be present. Such nonionic surfactant compounds include alkylmonoglycosides and alkylpolyglucosides. Alkylmonoglucosides and alkylpolyglucosides may be prepared by reacting a monosaccharide, or a compound hydrolyzable to a monosaccharide, with an alcohol such as a fatty alcohol in an acid medium. Various glucoside and polyglucoside compounds including alkoxylated glucosides and processes for making them are disclosed in U.S. Pat. Nos. 2,974,134; 3,219,656; 3,598,865; 3,640,998; 3,707,535, 3,772,269; 3,839,318; 3,974,138; 4,223,129 and 4,528,106 the contents of which are incorporated by reference.

One exemplary group of such useful alkylpolyglucoside surfactant compounds include those according to the formula:

R₂O—(CₙH₂ₙO)ᵣ—(Z)ₓ

wherein:
R₂ is a hydrophobic group selected from alkyl groups, alkylphenyl groups, hydroxyalkylphenyl groups as well as mixtures thereof, wherein the alkyl groups may be straight chained or branched, and which contain from about 8 to about 18 carbon atoms,
n has a value of 2-8, especially a value of 2 or 3;
r is an integer from 0 to 10, but is preferably 0,
Z is derived from glucose; and,
x is a value from about 1 to 8, preferably from about 1.5 to 5. Preferably the alkylpolyglucosides are nonionic fatty alkylpolyglucosides which contain a straight chain or branched chain C₈-C₁₅ alkyl group, and have an average of from about 1 to 5 glucose units per fatty alkylpolyglucoside molecule. More preferably, the nonionic fatty alkylpolyglucosides which contain straight chain or branched C₈-C₁₅ alkyl group, and have an average of from about 1 to about 2 glucose units per fatty alkylpolyglucoside molecule.

A further exemplary group of alkylglucoside nonionic surfactant compounds suitable for use in the practice of this invention may be presented by the following formula (A):

RO—(R₁O)_{y}-(G)ₓ-Z_{b} (A)

wherein:
R is a monovalent organic radical containing from about 6 to about 30, preferably from about 8 to 18 carbon atoms,
R₁ is a divalent hydrocarbon radical containing from about 2 to about 4 carbon atoms,
y is a number which has an average value from about 0 to about 1 and is preferably 0,
G is a moiety derived from a reducing saccharide containing 5 or 6 carbon atoms; and,
x is a number having an average value from about 1 to 5 (preferably from 1.1 to 2);
Z is O₂M¹,

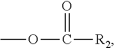

O(CH₂), CO₂M¹, OSO₃M¹, or O(CH₂)SO₃M¹;
R₂ is (CH₂)CO₂ M¹ or CH═CHCO₂M¹; (with the proviso that Z can be O₂M¹ only if Z is in place of a primary hydroxyl group in which the primary hydroxyl-bearing carbon atom, —CH₂OH, is oxidized to form a

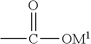

group)
b is a number of from 0 to 3x+1 preferably an average of from 0.5 to 2 per glycosal group;
p is 1 to 10,
M¹ is H⁺ or an organic or inorganic counterion, particularly cations such as, for example, an alkali metal cation, ammonium cation, monoethanolamine cation or calcium cation. As defined in Formula (A) above, R is generally the residue of a fatty alcohol having from about 8 to 30 and preferably 8 to 18 carbon atoms. Examples of such alkylglucosides as described above include, for example APG 325 CS Glucoside® which is described as being a 50% C₉-C₁₁ alkyl polyglucoside, also commonly referred to as D-glucopyranoside, (commercially available from Henkel KGaA) and Glucopon® 625 CS which is described as being a 50% C₁₀-C₁₆ alkyl polyglucoside, also commonly referred to as a D-glucopyranoside, (ex. Henkel).

Particularly preferred alkylpolyglucoside based nonionic surfactant compounds include those according to the following structure:

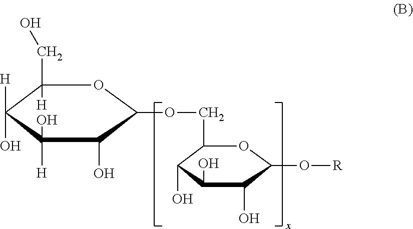

wherein:
R is an alkyl group, preferably a linear alkyl chain, which comprises C₈ to C₁₆ alkyl groups;
x is an integer value of from 0-3, inclusive.
   Examples of such alkylpolyglucoside compounds according to the aforesaid structure (B) include: where R is comprised substantially of C₈ and C₁₀ alkyl chains yielding an average value of about 9.1 alkyl groups per molecule (GLUCOPON 220 UP, GLUCOPON 225 DK); where R is comprised of C₈, C₁₀, C₁₂, C₁₄ and C₁₆ alkyl chains yielding an average value of about 10.3 alkyl groups per molecule (GLUCOPON 425N); where R is comprised substantially of C₁₂, C₁₄ and C₁₆ alkyl chains yielding an average value of about 12.8 alkyl groups per molecule (GLUCOPON 600 UP, GLUCOPON 625 CSUP, and GLUCOPON 625 FE, all of which are available from Cognis). Also useful as the alkylpolyglucoside compound is TRITON CG-110 (Union Carbide Corp. subsidiary of Dow Chemical). Further examples of commercially available alkylglucosides as described above include, for example, GLUCOPON 325N which is described as being a 50% C₉-C₁₁ alkyl polyglucoside, also commonly referred to as D-glucopyranoside (from Cognis). When present, the one or more more alkylglucoside (alkylglycoside) nonionic surfactant compounds do not exceed about 5% wt. of the inventive composition. The identity of and the preferred amounts of these compounds and preferred wt/wt ratios these compounds relative to other surfactant compounds are disclosed hereinafter in one or more of the Examples.

The treatment compositions are largely aqueous and thus comprise a predominant proportion of water. The treatment compositions preferably comprise at least 65% wt., preferably at least 75% wt. water. Concurrently however, the preferably comprises not more than 98% wt., more preferably not more than 96% wt. of water. The water may be from any suitable source including available tap water such as from a municipal water supply, reservoir or well, as well as deionized, demineralized, or distilled water. Deionized, demineralized, or distilled water a particularly preferred as reducing the quantity of undesirable impurities which may be present The water is provided in quantum sufficient (“q.s.”) in order to provide 100% wt. of the treatment composition.

In all embodiments the treatment compositions are acidic and exhibit a pH in the range of not more than 4.5 inclusive. Particularly preferred pH values are disclosed with reference to one or more of the following Examples.

The treatment compositions are desirably formed as free-flowing liquids having a viscosity of less than about 50 cPs, more preferably at less than about 10 cPs. as measured at room temperature (e.g. 20° C., 22° C. or 25° C.) using a standard quantitative laboratory techniques (.e.g., Brookfield RTV viscometer, using an appropriate spindle and speed setting.)

In addition to the essential constituents described hereinabove, the inventive compositions may further comprise one or more additional, albeit optional, constituents which may be used to provide a further technical and/or aesthetic attribute(s) to the treatment compositions. In certain preferred embodiments of the invention, one or more of the following recited optional constituents may be considered as essential constituents according to a particular preferred embodiment. Such optional constituents include one or more of: ancillary antimicrobial constituents, cosurfacants, thickeners, humectants, preservatives, antioxidants, solvents especially organic solvents, pH adjusting agents, pH buffers, chelating agents, fragrances, materials which provide an aromatherapy benefit, dyestuffs or colorants, and light stabilizers including UV absorbers. When present, the total amount of these various additives and adjuvants are those conventionally used in the field, and, for example, range from 0.01% to 75%, preferably 0.01%-50% wt. of the total weight of the treatment composition.

Any of the foregoing aspects of the invention may additionally include an ancillary antimicrobial constituent. Such may be at least one organic acid compound which provides an antimicrobial effect, other than the acids, salts and/or derivatives already present in the recited antimicrobial constituent. Such may be organic compounds which have less than about 12 carbon atoms, more preferably less than 10 carbon atoms and which comprise at least one moiety which impart acidity, preferably one or more groups selected from: most preferably are carboxyl groups (—COOH) which may provide a strong organic acid, and less preferably are hydroxyl groups (—OH), thiol group (—SH), enol groups (—C—C(OH)—), and phenols, which groups usually provide weaker organic acids than the preferred carboxyl groups (—COOH). The organic acid compounds may be aliphatic, aromatic, aryl, and may be substituted or unsubstituted with further functional groups. The foregoing moieties may be attached to any position of the carbon chain (or ring) of the antimicrobial constituent compound. Nonlimiting examples of such organic acids include: glycolic acid, salicylic acid, ascorbic acid, maleic acid, succinic acid, mandelic acid, dodecylbenzene sulfonic acid, propionic acid, gluconic acid, malic acid, benzoic acid, aspartic acid, acetic acid, oxalic acid, glutamic acid, adipic acid, hexanoic acid, octanoic acid, noanoic acid, decanoic acid, undecanoic acid, as well as mixtures of two or more of the foregoing. Of these organic acids, preferred are organic acids having a single carboxyl group, and especially preferred are alpha hydroxyl carboxylic acids. Of these, malic acid is preferred. It is to be further understood that one or more of these organic acids may also simultaneously function in adjusting the pH of the treatment compositions of which they form a part. However notwithstanding the foregoing, it is to be understood that one or more of the foregoing ancillary antimicrobial constituents may also be expressly excluded from the compositions of the invention. When present such an ancillary antimicrobial constituent may be present in any effective amount, advantageously forms from 0.001-5% wt. of a treatment composition of which it forms a part.

Preferably, known antimicrobial constituents based on antimicrobial free metal ions (e.g. Ag+, Cu2+, Zn2+), phenolic antimicrobial compounds (e.g. TRICLOSAN®, PCMX, TCC), and non-phenolic antimicrobial compounds (e.g. certain quaternary ammonium salts) are however excluded from the compositions of the invention.

The inventive compositions may include one or more cosurfactants which may be any surface active agents (or tensides,) other than those which are already present as an essential constituent according to one or more aspects of the invention. Such include anionic, nonionic, amphoteric surfactants as well as cationic surfactants which are present in an amount and/or which do not undesirably interact with one or more anionic compounds which may be present, e.g. by forming undesired complexes therebetween which diminish the surface activity of either such anionic and cationic compounds.

Non-limiting examples of anionic surfactants include alcohol sulfates and sulfonates, alcohol phosphates and phosphonates, alkyl ester sulfates, alkyl diphenyl ether sulfonates, alkyl sulfates, alkyl ether sulfates, sulfate esters of an alkylphenoxy polyoxyethylene ethanol, alkyl monoglyceride sulfates, alkyl sulfonates, alkyl ether sulfates, alpha-olefin sulfonates, beta-alkoxy alkane sulfonates, alkyl ether sulfonates, ethoxylated alkyl sulfonates, alkylaryl sulfonates, alkylaryl sulfates, alkyl monoglyceride sulfonates, alkyl carboxylates, alkyl ether carboxylates, alkyl alkoxy carboxylates having 1 to 5 moles of ethylene oxide, alkylpolyglycolethersulfates (containing up to 10 moles of ethylene oxide), sulfosuccinates, octoxynol or nonoxynol phosphates, taurates, fatty taurides, fatty acid amide polyoxyethylene sulfates, acyl glycerol sulfonates, fatty oleyl glycerol sulfates, alkyl phenol ethylene oxide ether sulfates, paraffin sulfonates, alkyl phosphates, isethionates, N-acyl taurates, alkyl succinamates and sulfosuccinates, alkylpolysaccharide sulfates, alkylpolyglucoside sulfates, alkyl polyethoxy carboxylates, and sarcosinates or mixtures thereof.

Further examples of anionic surfactants include alkyl-diphenyl-ethersulphonates and alkyl-carboxylates. Further anionic surfactants include salts (including, for example, sodium, potassium, ammonium, and substituted ammonium salts such as mono-, di- and triethanolamine salts) of soap, C₆-C₂₀ linear alkylbenzenesulfonates, C₆-C₂₂ primary or secondary alkanesulfonates, C₆-C₂₄ olefinsulfonates, sulfonated polycarboxylic acids prepared by sulfonation of the pyrolyzed product of alkaline earth metal citrates, C₆-C₂₄ alkylpolyglycolethersulfates (containing up to 10 moles of ethylene oxide); alkyl ester sulfates such as C₁₄₋₁₆ methyl ester sulfates; acyl glycerol sulfonates, fatty oleyl glycerol sulfates, alkyl phenol ethylene oxide ether sulfates, paraffin sulfonates, alkyl phosphates, isethionates such as the acyl isethionates, N-acyl taurates, alkyl succinamates and sulfosuccinates, monoesters of sulfosuccinate (especially saturated and unsaturated C₁₂-C₁₈ monoesters) diesters of sulfosuccinate (especially saturated and unsaturated C₆-C₁₄ diesters), acyl sarcosinates, sulfates of alkylpolysaccharides such as the sulfates of alkylpolyglucoside (the nonionic nonsulfated compounds being described below), branched primary alkyl sulfates, alkyl polyethoxy carboxylates such as those of the formula RO(CH₂CH₂O)ₖCH₂COO⁻M⁺ wherein R is a C₈-C₂₂ alkyl, k is an integer from 0 to 10, and M is a soluble salt-forming cation. Resin acids and hydrogenated resin acids are also suitable, such as rosin, hydrogenated rosin, and resin acids and hydrogenated resin acids present in or derived from tall oil.

Non-limiting examples of nonionic surfactants include include the polyoxyethylene ethers of alkyl aromatic hydroxy compounds, e.g., alkylated polyoxyethylene phenols, polyoxyethylene ethers of long chain aliphatic alcohols, the polyoxyethylene ethers of hydrophobic propylene oxide polymers, and the higher alkyl amine oxides.

Certain specific useful nonionic surfactants include primary and secondary linear and branched alcohol ethoxylates, such as those based on C₆-C₁₈ alcohols which further include an average of from 2 to 80 moles of ethoxylation per mol of alcohol. Examples include the Genapol® series of linear alcohol ethoxylates from Clariant Corp., Charlotte, N.C. The 26-L series is based on the formula RO(CH₂CH₂O)ₙH wherein R is a mixture of linear, even carbon-number hydrocarbon chains ranging from C₁₂H₂₅ to C₁₆H₃₃ and n represents the number of repeating units and is a number of from 1 to about 12. Useful also are secondary C₁₂-C₁₅ alcohol ethoxylates, including those which have from about 3 to about 10 moles of ethoxylation. Such are available in the Tergitol® series of nonionic surfactants (DOW Chemical, Midland, Mich.).

Certain preferred nonionic surfactants are fatty alkanolamides such those based ed on C₈-C₂₄ alkanols with an amine, e.g., coco monoethanolamide.

More specific nonionic surfactants include those in which the major portion of the molecule is made up of block polymeric C₂-C₄ alkylene oxides, with alkylene oxide blocks containing C₃ to C₄ alkylene oxides. Such nonionic surfactants, while preferably built up from an alkylene oxide chain starting group, can have as a starting nucleus almost any active hydrogen containing group including, without limitation, amides, phenols, and secondary alcohols.

A class of particularly useful nonionic block copolymer surfactants include those currently sold under the trademark of EcoSurf surfactants, (ex. DOW Chem. Co.) which materials are described to be block alkoxylates of nonlinear fatty alcohols, One such representative and preferred compound is represented by the following formula (F):

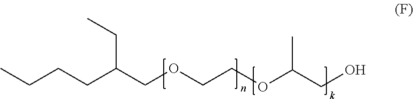

wherein the values of n and k are at least 1, and have values which are sufficient whereby a degree of aqueous miscibility is imparted to the comound.

Further useful nonionic surfactants include polyoxyethylene glycol ethers of C₈-C₂₄ fatty alcohols, such as steareth-2 and steareth-21, commercially available under the Brij tradename (ex. ICI).

One or more amphoteric surfactants may also be present as useful cosurfactants. Nonlimiting examples of such amphoteric surfactants include: alkylamine oxides, alkyamidopropyl amine oxides, alkyl betaines, alkyamidopropyl betaines, and sultaines.

Specific examples of alkyl amine oxides that may be used in the present invention include octyl amine oxide, decyl amine oxide, lauryl amine oxide, iso-dodecyl amine oxide, myristyl amine oxide, cetyl amine oxide, oleamine oxide, stearyl amine oxide, and palmitamine oxide. Specific examples of alkylamidopropyl amine oxides that may be used in the present invention include laurylamidopropyl amine oxide, cocamidopropyl amine oxide, stearamidopropyl amine oxide, germamidopropyl amine oxide.

Specific examples of alkyl betaines that may be used in the present invention include those which may be represented by the general formula:

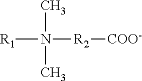

wherein R₁ is an alkyl group containing from 8 to 18 carbon atoms, or the amido radical which may be represented by the following general formula:

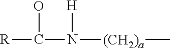

wherein R is an alkyl group having from 8 to 18 carbon atoms, a is an integer having a value of from 1 to 4 inclusive, and R₂ is a C₁-C₄ alkylene group. Non-limiting examples of useful betaines include octyl betaine, lauryl betaine, cocobetaine, cetyl betaine, oleyl betaine, and tallow dihydroxylethyl glycinate. Non-limiting examples of alkylamidopropyl betaines that may be used in the present invention include caprylamidopropyl betaine, capramidopropyl betaine, lauamidopropyl betaine, cocamidopropyl betaine, isostearamidopropyl betaine, wheatgermamidopropyl betaine, and coco/sunfloweramidopropyl betaine.

Specific examples of sultaines that may be used in the present invention include cocamidopropyl hydroxysultaine and lauryl hydroxysultaine.

While cationic surfactants which independently provide an antimicrobial benefits such as cationic surfactants based on quaternary ammonium compounds, e.g, alkylbenzyl dimethyl ammonium chloride, and dialkyl dimethyl ammonium chloride may be used in certain embodiments, usually however they are omitted from the compositions as such compounds would be expected to undesirably form complexes with one or more anionic surfactant compounds present, including those considered as essential in various aspects of the invention.

When present, such one or more cosurfactants may be present in any effective amount, but are advantageously present in amounts of about 0.01-10% wt., and more preferably about 0.1-5% wt., and especially preferably in amounts of about 0.25-3.5% wt. based on the total weight of the composition of which they forma part.

In certain embodiments, such as when the treatment compositions are formulated for use in the treatment of fibrous surfaces and textiles, such as carpets, upholstery and garments, one or more fluorosurfactants may be included. Such typically impart a degree of anti-resoiling and/or stain repellency to treated surfaces. Representative fluorosurfactants include those according to the formulae:

C₇F₁₅CO₂⁻NH₄⁺,

C₈F₁₇SO₂N(C₂H₅)(C₂H₄O)₇CH₃,

C₈F₁₇(C₂H₄O)₁₀H,

(C₄F₉SO₂)₂N⁻NH₄⁺,

C₄F₉SO₂N(CH₃)(C₂H₄O)ₙCH₃ (where n_{avg}^{˜}7), and

C₃F₇O(CF(CF₃)CF₂O)ₙCF(CF₃)CO₂⁻NH₄⁺ (where n_{avg}⁻13)

which are known to the art, and/or are commercially available such as FLURORAD surfactants (ex. 3M) or ZONYL surfactants (ex. DuPont).

Further useful fluorosurfactant include perfluoropropionates according to the following formula:

F(CF₂)ₙ—CH₂CH₂—S—CH₂CH₂—COO⁻X⁺ (A)

where:

n is an integer having a value of 6 to 12; and,

X⁺ is a salt forming counterion, preferably lithium.

Another exemplary further fluorosurfactant composition includes a perfluoroalkyl phosphate or salt thereof according to the following formula (B):

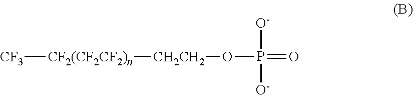

where:

n is an integer having a value of from 6 to 12.

The fluorosurfactant compositions according to the foregoing formulae (A) and (B) may be used singly, or may be used in a mixture. When used as a mixture, desirably the weight ratio of the perfluoropropionate to the perfluoroalkyl phosphate is in the range of from about 1:1 to 1:2. Such a mixture is presently commercially available as ZONYL® 7950.

Fluorinated acrylic polymers and salts thereof are also known to provide an antiresoiling benefit. An exemplary fluorinated acrylate copolymer is one which represented by the formula (C):

CF₃(CF₂)ₙCH₂OCOC(CH₃)═CH₂ (C)

wherein

n represents a value of from 6-8.

Such a fluorinated acrylate copolymer may be obtained commercially in a water based dispersion as SYNTRAN® 1575 (ex. Interpolymer Corporation, Canton, Mass.). It is to be understood that other fluorinated acrylate copolymer providing similar anti-resoiling benefits can be used as well.

Each of these fluorosurfactant compounds and/or fluorinated acrylic polymers may be used singly or in mixtures. When present, such one or more compounds or materials may be present in any effective amount, but are advantageously present in amounts of about 0.01-10% wt., and more preferably about 0.1-5% wt., and especially preferably in amounts of about 0.25-3.5% wt. based on the total weight of the composition of which they form a part.

Further anti-resoiling compositions may also be included, such as colloidal silica, aluminum oxides, styrene-maleic anhydride copolymer resins, polyvinylpyrrolidone, polyacrylates, polycarboxylates, modified cellulose polymers, vinyl acetate/maleic anhydride copolymer resins, cationic amines, aliphatic quaternary ammonium salts known to have anti-static properties, imidazoline salts as well as others known to the art. Preferred anti-resoiling compositions include polymers derived from monomers of acrylic acid, methacrylic acid, methacrylate, methyl-methacrylate, ethyl acrylate and maleic acid, as well as copolymers derived from the above monomers and olefin, in which the acrylic acid portion of the polymeric or copolymeric soil resist can be in the form of free acid, or a water soluble salt of acrylic acid (e.g., alkali metal salts, ammonium salts and amine salts). Such polymeric or copolymeric anti-resoiling compositions may be a mixture of acrylate polymers having a wide range of molecular weights. An exemplary anti-resoiling compound is a non-fluorinated, acrylic polymer compounds including those which may be represented by the following formula (D):

(—CH₂—CH(COOR)—)ₙ (D)

wherein

n is a value greater than 50.

Such a non-fluorinated acrylic polymer is presently commercially available in preparations including SYNTRAN® 1580, as well as SYNTRAN®1588 (ex. Interpolymer Corporation, Canton, Mass.). However, it is to be understood that other fluorinated acrylate copolymer providing similar anti-resoiling benefits can be used as well.

Such anti-resoiling compounds may be included in any effective amount, advantageously may be included in amounts of from 0-2% wt., but are desirably included in amounts of from 0.001% wt-1.5% wt.

The treatment compositions may include one or more chelating agents. Certain of these chelating agents may also provide a pH adjusting or pH buffering benefit as well. Exemplary useful chelating agents include those known to the art, including by way of non-limiting example; aminopolycarboxylic acids and salts thereof wherein the amino nitrogen has attached thereto two or more substituent groups. Examples of useful chelating agents include one or more of ethylene diamine tetra acetic acid (EDTA), diethylene triamine penta acetic acid (DTPA), ethane-1-hydroxy-1,1-diphosphonate (EHDP), ethylene diamine-N,N′-disuccinate (EDDS), nitrilo triacetic Acid (NTA), sodium imino disuccinate (IDS), ethylene glycol-bis-(2-aminoethyl)-N,N,N′,N′-tetra acetic acid (EGTA), methyl glycine diacetic acid (MGDA), N-(2-hydroxyethyl) ethylene diamine N,N′,N′-thacetic acid) (HEDTA), ethylene diamine tetra methylene phosphonic acid (EDTMP), diethylene thamine-penta-methylene phosphonic acid (DTPMP), glutamic acid-N,N-diacetic acid (GLDA), cyclohexane-1,2-diamine-N,N,N′,N′-tetra-acetic acid (CDTA), 1,3-propylenediamine tetra acetic Acid (PDTA), ethylene diamine triacetic acid (EDTA), L-hydroxy imino disuccinic acid (L-IDS), trisodium N-carboxyethyl imino succinate (CEIS), sodium tripolyphosphate (STP), thethylene tetramine hexaacetic acid (TTHA). Other preferred chelating agents are succinates, e.g., trisodium ethylene diamine disuccinate, tetra-sodium imino disuccinate, glutamic acid-N,N diacetic acid tetra sodium salt, 2-hydroxyethyl iminodiacetic acid, sodium salt (disodium ethanol diglycinate), tetrasodium 3-hydroxy-2,2 imino disuccinate, trisodium methylglycine diacetic acid, L-aspartate-N,N-diacetic acid tetrasodium salt. Particularly preferred chelating agents include acids and salts, especially the sodium and potassium salts of ethylenediaminetetraacetic acid, diethylenetriaminepentaacetic acid, N-hydroxyethylethylenediaminetriacetic acid, and of which the sodium salts of ethylenediaminetetraacetic acid may be particularly advantageously used. Further preferred chelating agents include those based on polyaspartic acid salts, which are considered to be more biodegradable than polycarboxylic acid salts. Non-limiting, and preferred examples of such preferred chelating agents include those presently commercially available as “Baypure” materials (ex. Lanxess), including Baypure CX100, a tetrasodium iminodisuccinate, and Baypure DS100, a sodium polyaspartate. Further nonlimiting examples of commercially available chelating agents include those marketed under the “Dissolvine” (ex. AkzoNobel). When present such one or more chelating agents usually comprise from 0.001-1% wt. of the inventive compositions.

The treatment compositions may and preferably do include a pH adjusting agent or pH buffer composition. Such compositions include many which are known to the art and which are conventionally used. By way of non-limiting example pH adjusting agents include phosphor containing compounds, monovalent and polyvalent salts such as of silicates, carbonates, and borates, certain acids and bases, tartarates and certain acetates. By way of further non-limiting example pH buffering compositions include the alkali metal phosphates, polyphosphates, pyrophosphates, triphosphates, tetraphosphates, silicates, metasilicates, polysilicates, carbonates, hydroxides, and mixtures of the same. Certain salts, such as the alkaline earth phosphates, carbonates, hydroxides, can also function as buffers. It may also be suitable to use buffers such materials as aluminosilicates (zeolites), borates, aluminates and certain organic materials such as gluconates, succinates, maleates, and their alkali metal salts. Desirably the compositions according to the invention include an effective amounts of an organic acid and/or an inorganic salt form thereof which may be used to adjust and maintain the pH or the compositions of the invention to the desired pH range. Particularly useful is sodium citrate which are widely available and which are effective in providing these pH adjustment and buffering effects. Such may be included in any effective amount, and advantageously are present in amounts of about 0.001-3% wt. of the inventive composition.

The treatment compositions may include one or more organic solvents. By way of non-limiting example exemplary useful organic solvents include those which are at least partially water-miscible such as low molecular weight alcohols, such as, C₁-C₆ monohydric alcohols including, e.g. ethanol, propanol, isopropanol, and butanol; glycols, e.g., ethylene glycol, propylene glycol, hexylene glycol; water-miscible ethers, e.g. diethylene glycol diethylether, diethylene glycol dimethylether, propylene glycol dimethylether; water-miscible glycol ethers, e.g. propylene glycol monomethylether, propylene glycol mono ethylether, propylene glycol monopropylether, propylene glycol monobutylether, ethylene glycol monobutylether, dipropylene glycol monomethylether, diethyleneglycol monobutylether; lower esters of monoalkylethers of ethylene glycol or propylene glycol, e.g. propylene glycol monomethyl ether acetate, and mixtures thereof. Glycol ethers having the general structure Rₐ—R_{b}—OH, wherein Rₐ is an alkoxy of 1 to 20 carbon atoms, or aryloxy of at least 6 carbon atoms, and R_{b} is an ether condensate of propylene glycol and/or ethylene glycol having from one to ten glycol monomer units are also useful. When present they may be used in any effective amount, and advantageously are present from 0.01% wt., to about 10% wt. of the said compositions.

The treatment compositions may include a humectant, particularly when they are formulated for use in topical applications to the skin, hair or other dermal surfaces including hair, including as a skin treatment composition which imparts an antimicrobial benefit to the skin (particularly the hands) and/or as a hand wash or body wash composition. Preferred humectants include sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine. Further useful humectants include sodium 2-pyrrolidone-5-carboxylate, guanidine; glycolic acid and glycolate salts (e.g. ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; and, panthenol. Still further humectants include polyols e.g., linear and branched chain alkyl polyhydroxyl compounds having a boiling point of at least 120° C., preferably at least 140° C. and yet more preferably at least 155° C. at 760 mm Hg (=1 atmosphere) such as: propylene glycol, polyethylene glycol, glycerine and sorbitol. Further exemplary hydrocarbons which may also serve as humectants are those having hydrocarbon chains anywhere from 12 to 30 carbon atoms, particularly, mineral oil, petroleum jelly, squalene and isoparaffins. Particularly preferred polyols useful as humectants are glycerine, glycerol, sorbitol as well as mixtures thereof. Glycerine and glycerol are particularly preferred for their use as humectants. When present, such are advantageously included in amounts of from about 0.01-5% wt. of the treatment composition.

The treatment compositions may include a thickener constituent, non-limiting examples of which include one or more of polysaccharide polymers selected from cellulose, alkyl celluloses, alkoxy celluloses, hydroxy alkyl celluloses, alkyl hydroxy alkyl celluloses, carboxy alkyl celluloses, carboxy alkyl hydroxy alkyl celluloses, naturally occurring polysaccharide polymers such as xanthan gum, guar gum, locust bean gum, tragacanth gum, or derivatives thereof, polycarboxylate polymers, polyacrylamides, polyacrylate cross-polymer thickeners, clays, and mixtures thereof. Such may be included in any effective amount, and the amount depends upon the nature of the selected thickener and upon the final viscosity required of the treatment composition. When present, such is advantageously included in amounts of from about 0.001-5% wt. of the treatment composition.

The viscosity of the treatment composition may also be thickened by the addition of metal salts, e.g. sodium chloride or other inorganic salts to the compositions which may interact with one or more of the surfactant compounds, present, e.g. n-acyl sarcsonate compounds. Again, such may included in any effective amount to achieve a desired increase in viscosity.

The treatment compositions may include a fragrance constituent, which may be based on natural and synthetic fragrances and most commonly are mixtures or blends of a plurality of such fragrances, optionally in conjunction with a carrier such as an organic solvent or a mixture of organic solvents in which the fragrances are dissolved, suspended or dispersed. The inclusion of such a fragrance constituent may be desirable as such may provide a degree of odor masking. Such is advantageously present in amounts of up to about 1% wt., preferably are present in amounts of from about 0.00001% wt. to about 0.5% wt., based on the total weight of the composition of which it forms a part.

The treatment compositions may include one or more essential oils which are selected to provide a so-called “aromatherapy benefit” to the user. Such are advantageously used when the treatment compositions are formulated as topical compositions, e.g, hand wash compositions or body wash compositions. Such one or more essential oils providing an aromatherapy benefit are present in an amount about 0.00001 wt. % to about 1 wt. %, but preferably to about 0.5 wt. % of the total weight of the composition. It is to be understood that these one or more essential oils providing an aromatherapy benefit may be used with our without the optional fragrancing constituent recited previously and may be used wholly or partially in place of said fragrancing constituent.

The treatment compositions may include one or more colorants, e.g. dyes, pigments, and the like. Preferred are pigments and/or dyes include those approved for use by an appropriate government body or agency, such as FD&C dyes used in the U.S.A. Advantageously one or more colorants may be added in amounts of about 0.001% wt. to about 0.1% by weight, based on the total weight of the composition of which the colorant(s) forms a part.

Particularly when formulated as a topical composition, the treatment compositions may optionally include one or more vitamins, vitamin derivatives and/or vitamin precursors, nonlimiting examples of which include Vitamin A, D, E, panthenol, niacinamide, and retinyl palmitate. When included, such one or more such vitamins, derivatives and/or precursors thereof may be present in a therapeutically effective amount, which may vary upon the identity of the particular such material. Advantageously, amounts of from 0.0001-1% wt., based on the total weight of the treatment compositions are expected to be effective.

Especially when formulated as a topical treatment composition, the inventive compositions may include one or more antioxidant constituents. Examples of antioxidants include but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, glutathione, and ascorbic acid and ascorbic acid derivatives (e.g., ascorbyl palmitate and ascorbyl polypeptide). Oil-soluble antioxidants suitable for use in the feminine intimate cleansing compositions of this invention include, but are not limited to, butylated hydroxytoluene, retinoids, tocopherols e.g., tocopherol acetate, tocotrienols, and ubiquinone. Natural extracts containing antioxidants suitable for use in the topical compositions of this invention, include but not limited to, extracts containing flavonoids and isoflavonoids and their derivatives, extracts containing resveratrol and the like. Examples of such natural extracts include grape seed, green tea, pine bark, propolis, and the like. When present the total amount of such antioxidants are usually not in excess of 5% wt. and preferably are present from 0.001-2.5% wt.

The treatment compositions may include a particulate material which may be desirably included when the treatment composition is formulated as a topical scrub or topical washing composition. Such may be any particulate material which is a solid at room temperature, which does not deleteriously react chemically with the balance of the constituents of the inventive composition. Such a particulate material provide for a controlled and desirably degree of skin abrasion when the feminine intimate cleansing composition is applied, and especially where it is rubbed into the skin e.g, to provide a cleaning and/or exfoliating benefit. Non-limiting examples of such materials include: inorganic particulates, polymeric organic particulates, carbonates, hollow silica microspheres, glass microcapsules, ceramic microcapsules, inorganic pigments, crystalline and microcrystalline waxes derived from plants, mineral oils or petroleum, hollow polymer microspheres, starches, alginates, organic dyestuffs or pigments, and mixtures thereof. Preferably such have an average particle size in the range of about 1-100 microns, more preferably about 5-50 microns, as measured along the widest dimension of the particulate. Such may be present in any effective amount, preferably from 0.01-5% wt. of the treatment composition of which it forms a part.

One or more inert fillers in the form of powders may also be present in the treatment compositions. By way of non-limiting examples these powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof. Such may also provide a viscosity building or thickening benefit. When present such fillers may be present in amounts of up to about 5% wt., preferably are present in amounts of from about 0.001% wt. to about 5% wt. based on the total weight of the treatment composition of which it forms a part.

Especially when formulated as a topical composition, the treatment compositions may include a cationic Polyquaternium-type polymer. Such materials are well known to the art of topical compositions and are described in the literature, particularly in the International Cosmetic Ingredient Dictionary and Handbook, Volume 2 (9^{th} Edition, 2002), at pages 1311-1319. Other polyquaternium compounds although not specifically elucidated here may also be utilized in the present inventive compositions. Preferably any included polyquaternium compounds do not react to an undesirable degree with any anionic compounds, particularly any anionic surfactant which may be present. When included such one or more cationic polyquaternium-type polymers are advantageously present in amounts of from about from 0.001-5% wt., preferably in amounts from 0.01-2% wt., based on the total weight of the treatment composition of which they form a part.

The treatment compositions may include one or more opacifiers. Such are materials which are typically emulsions, dispersions or suspensions of a water insoluble polymer or copolymer in an carrier. The carrier may be aqueous, an aqeueous/organic solvent mixture or organic solvent. The latex may be based on a homopolymer, or on copolymer formed from styrene, alpha-methylstyrene, divinylbenzene, acrylic acid or C₁-C₂₀ esters thereof, methacrylic acid or C₁-C₂₀ esters thereof, (meth)acrylamide, maleic acid, vinyl acetate, crotonic acid, vinyl neodecanoate and butenoic acid. Particularly preferred latexes useful in the present invention are latexes presently commercially available under the trademark ACUSOL (ex. Rohm & Haas Inc.). When present in a composition, the latex may be present in amounts of up to about 5% wt., preferably are present in amounts of from about 0.001% wt. to about 3% wt. of the compositions of which they form a part.

The treatment compositions may include one or more light stabilizers and/or UV absorbers, particularly when such are formulated as a topical composition. Exemplary and preferred such materials which are presently commercially available include one or more of: CIBAFAST H liquid, described to be sodium benzotriazolyl butylphenol sulfonate with Buteth-3 and tributyl citrate; TINOGARD HS described to be sodium benzotriazolyl butylphenol sulfonates; TINOGARD AS described to be bumetrizole. When present, the one or more light stabilizers and/or UV absorbers may be included in any effective amount; advantageously form from 0.0001-1% wt. of the treatment composition of which it forms a part.

The treatment compositions may include one or more preservatives, e.g. compounds such as: phenoxyethanol, ethylhexylglycerin, dicaprylyl glycol, formaldehyde solutions, parabens, pentanediols, benzoic acid, sorbic acid, benzyl alcohol, as well as mixtures of two or more of the foregoing. Also useful are commercially available preservative preparations include a mixture of 5-chloro-2-methyl-4-isothiazolin-3-one and 2-methyl-4-isothiazolin-3-one marketed under the trademark KATHON CG/ICP as a preservative composition (ex. Rohm and Haas Inc.), and a mixture of mixture of caprylyl glycol and ethylhexylglycerin supplied as Sensivia SC10 (ex. Schülke+Mayr). Further useful preservative compositions include KATHON CG/ICP II (ex. Rohm and Haas Inc.), PROXEL (ex. Zeneca), SUTTOCIDE A (ex. Sutton Laboratories) as well as TEXTAMER 38AD (ex. Calgon Corp.) Preferred preservative compositions are based on one or more constituents which are considered to be “natural” or “organic” materials by relevant standards setting organizations (e.g., ECOCERT, NaTrue, BDIH) such as blends of blend of benzyl alcohol, benzoic acid and sorbic acid which in even very low concentration (about 1% wt. or less) provide a long term product preservative effect. When present the preservative is included in any amount found to be effective in retarding or inhibiting the growth of undesired microorganisms in the treatment compositions, and is advantageously present in amounts of up to about 1.5% wt., preferably are present in amounts of from about 0.00001% wt.-1% wt., based on the total weight of the composition of which it forms a part.

While not wishing to be bound by the following hypothesis it is nonetheless believed that by careful selection of the surfactants present in the compositions, and in particular the required inclusion of the specific recited systems of surfactants according to a particular recited aspect, that the compositions of the present invention may achieve good antimicrobial results against various challenge organisms when tested according to the demanding protocols of one or more of: (i) AOAC Official Method 961.02 Germicidal Spray Products as Disinfectants, including also when modified for use with towelettes according to EPA Series 810 guidelines (OCSPP 810.2200: Disinfectants for Use on Hard Surfaces—Efficacy Data Recommendations), (ii) AOAC Official Method 955.14, 955.15 (preferably against S. aureus and/or S. enterica); (iii) European Standard EN 1276:2009—Chemical Disinfectants and Antiseptics—Quantitative suspension test for the evaluation of bactericidal activity of chemical disinfectants used in the food, industrial, domestic and institutional areas; (iiii) European Standard Surface Test EN 13697:2001—Chemical disinfectants and antiseptics—Quantitative non-porous surface test for the evaluation of bactericidal and/or fungicidal activity of chemical disinfectants used in food, industrial, domestic and institutional areas; (v) ASTM E1153—Standard Test Method for Efficacy of Sanitizers Recommended for Inanimate Non-Food Contact Surfaces, and/or other standardized testing protocols. Preferred embodiments of the treatment compositions provide a broad spectrum antimicrobial benefit. It is hypothesized that the specific selection of the surfactants, further in conjunction with the antibacterial constituent work cooperatively, and it is believed that these surfactants aid in potentiating the activity of the antimicrobial constituent (e.g., lactic acid a/o citric acid a/o salts thereof) such the compositions achieve broad spectrum antimicrobial efficacy (preferably are concurrently effective against at least both gram positive and gram negative bacteria) that it may be used in relatively reduced amounts as compared to other and different systems of surfactants (and optionally further constituents as well, e.g. organic solvents) in order to achieve corresponding levels of antibacterial activity as may be demonstrated by one or more of the foregoing tests. These results are believed to be in part dependent not only on the specific surfactants according to a specific system of a specific inventive aspect, but preferably also that the relative weight ratios of the individual surfactants are provided within specific wt/wt ratios, and possibly also with respect to the relative amount of the antimicrobial constituent which is also present. Such ratios vary amongst the different recited aspects of the invention.

According to the first aspect of the invention the wt/wt ratio (or parts) of the (a) secondary alkane sulfonate surfactant compound(s): (b) N-acyl sarcosinate compound(s): (c) anionic hydrotrope surfactant compound(s), viz. (a):(b):(c) is 2-6:0.1-2:1, preferably is 3.5-5:0.2-2:1, with particularly preferred ratios being demonstrated with respect to one or more of the Example compositions.

According to the second aspect of the invention the wt/wt ratio (or parts) of the (a) secondary alkane sulfonate surfactant compound(s), and the (c) anionic hydrotrope surfactant compound(s), viz., (a):(c) is 0.2-20:1, preferably is 0.25-15:1, with particularly preferred ratios being demonstrated with respect to one or more of the Example compositions.

According to the first variant of the third aspect of the invention the wt/wt ratio (or parts) of the (a) alkyl sulfonate surfactant compound(s) selected from alkylbenzene sulfonate compound(s) secondary alkyl sulfonate surfactant compound(s): (b) alkylglycoside nonionic surfactant compound(s): (c) aromatic hydrotrope compound(s), viz, the ratio (a):(b):(c) is desirably 0.5-2.5:0.2-0.8:1, preferably 1-2:0.25-0.7:1, with particularly preferred ratios being demonstrated with respect to one or more of the Example compositions.

According to the second variant of the third aspect of the invention the wt/wt ratio (or parts) of the (a) alkyl sulfonate surfactant compound(s) selected from an alkylbenzene sulfonate compound and a secondary alkyl sulfonate surfactant compound(s): (b) alkylglycoside nonionic surfactant compound(s): (c) aromatic hydrotrope compound(s): (d) an N-acyl sarcosinate compound(s), viz., the ratio of (a):(b):(c):(d), is desirably 0.5-2.5:0.2-0.9:1:0.25-1, preferably 1-2:0.25-0.7:1:0.5, with particularly preferred ratios being demonstrated with respect to one or more of the Example compositions.

According to the fourth aspect of the invention the wt/wt ratio (or parts) of the (a) alkylbenzene sulfonate surfactant compound(s): (b) anionic aromatic hydrotrope compound(s), viz, the ratio (a):(b) is desirably is 1-3.5:1, preferably is 1.5-3.25:1 with particularly preferred ratios being demonstrated with respect to one or more of the Example compositions.

According to the first variant of the fourth aspect of the invention the wt/wt ratio (or parts) of the (a) alkylbenzene sulfonate surfactant compound(s): (b) anionic aromatic hydrotrope compound(s): (c) one or more further anionic surfactant(s) other than (a) and (b), viz. the ratio of (a):(b):(c), is desirably 0.5-1:1:5-40, preferably about 0.75-0.85:1:7.5-32.5, with particularly preferred ratios being demonstrated with respect to one or more of the Example compositions.

According to the second variant of the fourth aspect of the invention the wt/wt ratio (or parts) of the (a) alkylbenzene sulfonate surfactant compound(s): (b) anionic aromatic hydrotrope compound(s): (c) one or more further anionic surfactant(s) other than (a) and (b): (d) alkylglucoside nonionic surfactant compound(s), viz. the ratio of (a):(b):(c):(d), is desirably 0.5-1:1:5-40:2.5-7.5, preferably about 0.75-0.85:1:7.5-32.5:4-6, with particularly preferred ratios being demonstrated with respect to one or more of the Example compositions.

According to the fifth aspect of the invention the wt/wt ratio (or parts) of the (a) secondary alkane sulfonate surfactant compound(s): (b) n-acyl sarcosinate compound(s): (c) anionic hydrotrope surfactant compound(s), viz. the ratio of (a):(b):(c), is desirably 2.5-7.5:1-2.5:1, preferably is 6-7: 1-2:1, with particularly preferred ratios being demonstrated with respect to one or more of the Example compositions.

Particularly preferred amounts, and preferred relative ratios of required surfactants a/o compounds in the foregoing recited “systems” according to specific aspects of the invention, as well as the relative amounts of the antimicrobial constituent relative to a particular system, are disclosed with reference to one or more of the Examples set forth below.

It is suspected that the use of the specific recited surfactant compound(s) in specific wt/wt ratios may exhibit a synergy in boosting or potentiating the efficacy of the antimicrobial constituent, such that the amount which would otherwise required to meet a desired degree of antimicrobial efficacy can be reduced, and/or that the pH of such compositions may be formulated to be less acidic than would be otherwise required to meet a desired degree of antimicrobial efficacy as compared to otherwise like treatment compositions wherein one or more of the required surfactants according to a particular inventive aspect are omitted, and/or wherein one or more of the stated required surfactants is present in an amount outside of the specific wt/wt ratios disclosed herein. Additionally or alternatively, with the use of the specific required surfactants of a system according to one or more of the inventive aspects, particularly and preferably when within the specific wt/wt ratios disclosed herein the amount of the antimicrobial constituent may be reduced while still providing a desired degree of antimicrobial efficacy, and particularly preferably demonstrating a broad spectrum antimicrobial efficacy against both gram positive and gram negative species of bacteria.

Preferably the treatment compositions of the invention exhibit an antimicrobial effect (preferably sanitizing or disinfecting) and preferably a broad spectrum antimicrobial effect, as demonstrated according to known test protocols described previously and/or described hereinafter with reference to the Examples.

The compositions of the invention may also exhibit a virucidal benefit as well, such as against enveloped viruses, e.g., SARS, coronavirus and/or influenza. such as may be determined by ASTM E1052—Standard Test Method to Assess the Activity of Microbicides against Viruses in Suspension, and/or ASTM E1053—Standard Test Method to Assess Virucidal Activity of Chemicals Intended for Disinfection of Inanimate, Nonporous Environmental Surfaces, and/or other appropriate standardized protocols.

The treatment compositions may be packaged in any suitable container for storage and/or dispensing as may be desired or required. Examples of such dispensing containers include pressurized containers such as aerosol canisters which include a quantity of the composition as well as a propellant, as well as nonpressurized flasks or vessels which are equipped with a manually-pumpable spray head, e.g, a manually pumpable trigger spray head, through which can be dispensed the animal treatment compositions. Further exemplary dispensing container are nonpressurized flasks or vessels provided with a removable cap or a flow-directing nozzle from which can be poured quantities of the animal treatment compositions.

When provided in a pressurized or pressurizable container, e.g., an aerosol canister supplied with a conventional valve, the compositions of the invention would additionally require a propellant, which may be chosen from those presently used in the art and include, for example, compressed gases such as carbon dioxide, compressed air, or nitrogen, as well as C₁-C₁₀ hydrocarbons, such as n-propane, n-butane, isobutane, n-pentane, isopentane, dimethyl ether and blends thereof may be used. When utilized, an amount of about 0.5-25% wt. of propellant is added to a 100% wt. animal treatment composition previously formed.

The treatment compositions may be used without any further dilution in water, or may be used in a diluted form, particularly wherein the treatment compositions are diluted with water on a wt/wt (or vol/vol) basis in the following ratios of treatment composition:water of 1:1-128, preferably 1:1-64, more preferably 1:4-64, and particularly preferably 1:16-32.

In certain preferred embodiments the treatment compositions are provided with a carrier substrate, e.g., a as a wipe type product or wipe article, wherein the final form of the consumer product is a preimpregnated pad or wipe, (which may be wet or dry) containing a quantity of the treatment composition. Useful as pads or wipes suitable are essentially any material which is effective as a carrier for a quantity of the treatment composition. Advantageously, fibrous materials formed of natural and/or synthetic fibers may be used. The nonwoven fabrics may be a combination of wood pulp fibers and textile length synthetic fibers formed by well known dry-form or wet-lay processes. Synthetic fibers such as rayon, nylon, orlon and polyester as well as blends thereof can be employed. Such may be woven, or nonwoven wipes, or pads. Such can be resin bonded, hydroentangled, thermally bonded, meltblown, needlepunched, or any combination of the former. The substrate of the wipe may also be a film forming material such as a water soluble polymer. Such film substrates may be sandwiched between layers of fabric substrates and heat sealed to form a useful substrate. The films themselves can be extruded utilizing standard equipment to devolatilize the blend. Casting technology can be used to form and dry films, or a liquid blend can be saturated into a carrier and then dried in a variety of known methods.

Although the pads or wipes used in a wipe type product or wipe article may have little or no content of cellulosic materials, preferred wipe type products and wipe articles are those which are predominantly (viz, in excess of 50%) based on cellulosic fibers, particularly, those based on natural fiber sources such as cotton, or pulp are preferably used due to their efficacy, ready availability, and low cost. More preferably the preferred pads or wipes contain at least 75%, and in order of increasing preference, at least 80%, 85%, 90%, 95%, 97%, 98%, 99%, and 100% based on cellulosic fibers.

Also useful as a wipe type product or wipe article are closed cell, or open celled foams such as polyurethane foams as well as regenerated cellulose foams, which may also be referred to as sponges.

Any of the foregoing wipe type product or wipe article may include a laminate layer, such as a liquid impervious layer which may be useful in limiting the wicking of the treatment composition to a part or surface of the pad, wipe or sponge.

Preimpregnated wipe type product or wipe article may be provided at any useful loading ratio of treatment composition:non-impregnated pad or wipe, but preferably such are loading ratio is the range of about 0.1-5 wt./wt., preferably 0.5-5:1 wt./wt.

Dry preimpregnated pads or wipes can mean made by simply applying a suitable amount of the treatment composition, and thereafter allowing it to dry, in which case the initial loading ratio prior to any drawing is considered as being effective loading ratio of treatment composition:non-impregnated pad or wipe.

Such a wipe type product or wipe article product may be supplied in any suitable container for storage and/or dispensing which maybe is a dispense a single, or a plurality of preimpregnated pads or wipes. Single preimpregnated pads or wipes are conveniently provided in breachable envelopes or pouches formed from barrier materials, such as polymeric films, foils, and metallized polymer films, and/or, co-laminates of one or more of the foregoing such barrier materials. Such barrier materials aid in retaining the moisture content of the preimpregnated pad or wipe prior to use. A plurality of preimpregnated pads or wipes a be provided in a sealable container which may be used to dispense one or more pads or wipes as desired, and thereafter closed by consumer or user. Non-limiting examples thereof include polymeric tubs, flasks, and resealable pouches.

The treatment compositions may be used to treat animate surfaces, e.g, topical surfaces including parts of the human body, as well as parts of non-human bodies including those of pets, e.g, canine and feline pets. The treatment compositions may be used to treat inanimate surfaces, e.g, hard surfaces and soft surfaces, e.g., clothing, upholstery, drapes, carpets, and the like, especially including those which have come into contact with non-human animals, such as cats, dogs, other non-human mammals and reptiles. Non-limiting examples of hard surfaces are generally non-porous materials such as surfaces composed of refractory materials such as: glazed and unglazed tile, brick, porcelain, ceramics as well as stone including marble, granite, and other stones surfaces; glass, metals, plastics e.g. polyester, vinyl, fiberglass, Formica®, Corian® and other hard surfaces known to the industry. Non-limiting examples of soft surfaces include generally porous materials such as carpets, rugs, upholstery, curtains and drapes, fabrics, textiles, garments, and the like. The quantity of the treatment compositions to be used in such application varies and depends upon the nature of a stain or surface being treated using the treatment compositions, and the purpose of the treatment, e.g., a desired degree of cleaning and/or antimicrobial benefit sought. Advantageously about 10-35 grams/m², preferably about 22-25 grams/m² are applied to a surface to be treated when the composition is dispensed from a manually pumpable trigger spray. When the compositions are delivered to treated surface from a wipe article, (preferably a non-woven wipe substrate), advantageously about 5-15 grams/m², preferably about 9 grams/m² are applied to a surface. Such an application and/or treatment step may be repeated as necessary.

Certain preferred embodiments of the invention are packaged vendible articles, which includes a quantity of a treatment composition. Such may be in any of the foregoing formats, including sprayable liquids and wipes. Preferred embodiments of the treatment composition include: topical compositions, hand wash compositions, body wash compositions, hair treatment compositions, pet treatment compositions for the direct treatment of the bodily surface of a non-human animal, e.g, canine species, feline species, hard surface treatment compositions particularly for the cleaning and/or antimicrobial (disinfecting, sanitizing) treatment of hard surfaces and/or soft surfaces. A particularly preferred product format is a treatment composition which is used in the cleaning and/or antimicrobial treatment of a hard surface wherein a subsequent aqueous rinsing step is not required; such is particularly relevant to food preparation surfaces, e.g, kitchen surfaces such as countertops, lavatory surfaces including the surfaces of toilets, toilet seats, bidets, washstands, sinks, showers, and bathtubs. Omission of such a post-application aqueous rinse may provide for a residual antimicrobial benefit to be present on such treated surfaces.

The following examples below illustrate exemplary formulations as well as preferred embodiments of compositions, as well as wipe articles according to the certain aspects of the invention. It is to be understood that these examples are provided by way of illustration only and that further compositions and articles may be produced in accordance with the teachings of the present invention.

### EXAMPLES

A number of compositions according to the present invention were produced according to the invention described below with reference to Tables 1A, 1B, 1C, 1D and 1E. In those compositions, the identified compounds/constituents were supplied by raw materials identified on Table 2 which one or more raw materials may have had less than 100% wt. “actives” content, or may have comprised 100% wt. “active” of the named compound/constituent. These materials were used “as supplied” from their respective supplier. Additionally, to each of the compositions was included deionized water in “quantum sufficient” (q.s.) in order to provide 100 parts by weight of the specific composition, and in some instances an amount of one or more pH adjusting constituents, e.g., acids and/or bases, such as sodium hydroxide, may have been added in order to establish a desired pH for the composition, which similarly required a “q.s.” amount.

Compositions identified by a digit with a prepended letter “E” are examples according to the invention, whereas compositions identified by a digit with a prepended letter “C” are comparative examples.

The compositions of Table 1A illustrate inventive compositions according to the first recited aspect of the invention, and the compositions of Table 1B illustrate inventive compositions according to the second recited aspect of the invention. The compositions of the first and second recited aspects of the invention may be used in a variety of product formats, and are particularly well suited when formulated as topical compositions particularly as hand wash compositions and body wash composition. These compositions provide an antimicrobial benefit to topical surfaces upon which they are used and may also provide a concurrent cleaning benefit as well.

The compositions of Table 1C disclose embodiments of compositions of the third aspect of the invention, with the first part of the table “Table 1C (Spray)” providing formulations suitable to use in a spray dispenser, and the second part of the table “Table 1C (Wipes)” providing formulations which had been absorbed at the indicated loading ratio onto a dry wipe, and thereafter tested It is to be understood however that the liquid compositions of the said first part of the table may also be used with a carrier substrate, e.g. a wipe, and similarly the compositions on the second part of the table may be used without a carrier substrate, e.g. a liquid or spray. The compositions and wipe articles are particularly suited for use as a pet care composition, wherein the treatment composition, either applied as a liquid or via a carrier substrate is used to contact the surface of non-human animals, e.g, cats and dogs, as well as other mammalian species, in order to provide an antimicrobial benefit, and also an optional cleaning benefit thereto. The compositions and wipe articles are also well suited to treat inanimate surfaces.

The compositions of Table 1D also illustrate compositions according to the two variants of the recited fourth aspect of the invention. These compositions are particularly useful in providing an antimicrobial benefit to a treated inanimate surface and do not require that they be rinsed off with a subsequent aqueous rinse, as is frequently recommended for use with many current commercially available hard surface disinfecting or sanitizing preparations. Such compositions are also considered to be “food safe” and do not necessitate a subsequent aqueous rinse of the treated hard surface. Compositions of the fourth aspect of the invention also provide a cleaning benefit to treated surfaces, particularly hard surfaces. Compositions of the fourth aspect of the invention are particularly well suited in the antimicrobial treatment of kitchen and lavatory surfaces, particularly hard surfaces.

The compositions of Table 1E illustrate inventive compositions of the fifth aspect of the invention wherein are provided acidic compositions having a pH of about 4.5 and less and in which an antimicrobial constituent selected salicylic acid and lactic acid are omitted, but which compositions include sufficient citric acid to adjust to the indicated target pH and which further includes an antimicrobially effective amount of a ternary anionic surfactant system which necessarily comprises one or more of each of: (a) a secondary alkane sulfonate surfactant compound(s), (b) n-acyl sarcosinate compound(s) and (c) an anionic hydrotrope surfactant compound(s) is present. The foregoing composition may be used in a formulation which adds further optional constituents, but which omits the need for salicylic acid and lactic acid, or the inclusion of known antimicrobial constituents those based on antimicrobial free metal ions (e.g. Ag+, Cu2+, Zn2+), phenolic antimicrobial compounds (e.g. TRICLOSAN®, PCMX, TCC), and non-phenolic antimicrobial compounds (e.g. certain quaternary ammonium salts).

TABLE 1A

| | E1 | E2 | E3 | E4 |
|---|---|---|---|---|
| secondary alkane | 12.0 | 12.5 | 12.0 | 6.66 |
| sulfonate (60%) | | | | |
| lauroyl sarcosinate (30%) | 10.0 | 10.0 | 9.5 | 0.7 |
| cumene sulfonate (40%) | 4.0 | 3.0 | 3.0 | 2.5 |
| lauryl ether sulfate 2EO | — | — | — | 7.1 |
| (70%) | | | | |
| ammonium lauryl sulfate | — | — | — | 18.03 |
| (25%) | | | | |
| alkyl benzene sulfonic | — | — | — | — |
| acid | | | | |
| coco monoethanolamide | — | 1..0 | — | 1.0 |
| cocoamidopropyl betaine | — | — | 4.0 | — |
| (30%) | | | | |
| salicylic acid | 0.4 | 0.3 | 0.3 | — |
| lactic acid (90%) | — | — | — | 2.5 |
| tartaric acid | 0.5 | — | 0.5 | — |
| citric acid | 0.5⁺ | — | 0.55 | — |
| citric acid (50%) | — | *** | *** | 0.1 |
| sodium lactate | — | — | 0.4 | — |
| PCMX | — | — | 0.175 | — |
| Polyquaternium-7 | — | — | 0.1 | — |
| guar gum1 | — | 0.1 | — | 0.1 |
| glycerine | 1.0 | 1.0 | 0.9 | 1.0 |
| propylene glycol | 1.0 | 1.0 | 1.0 | 1.0 |
| sodium hydroxide (30%) | — | — | **** | — |
| sodium hydroxide (50%) | — | — | — | 0.1 |
| sodium chloride (20%) | — | ** | ** | — |
| sodium citrate dihydrate | — | — | 0.6 | — |
| tetrasodium EDTA | 0.1 | 0.1 | 0.1 | 0.1 |
| cellulose thickener | — | — | 0.6 | — |
| preservative | — | 0.02 | 0.02 | 0.02 |
| di water | q.s. | q.s. | q.s. | q.s. |
| pH | 3.51-3.57 | — | — | 4.19 |
| Viscosity (cP) | — | — | — | 4000-8000 |

relative ratios: (wt/wt)

| | | | | |
|---|---|---|---|---|
| secondary alkane | 4.5 | 6.25 | 6.0 | 4.0 |
| sulfonate | | | | |
| lauroyl sarcosinate | 1.88 | 2.5 | 2.38 | 0.21 |
| hydrotrope | 1.0 | 1.0 | 1 | 1.0 |

| | E5 | E6 | E7 | E8 |
|---|---|---|---|---|
| secondary alkane sulfonate | 12.0 | 6.66 | 6.66 | 6.66 |
| (60%) | | | | |
| lauroyl sarcosinate (30%) | 10.0 | 0.7 | 0.7 | 0.7 |
| cumene sulfonate (40%) | — | 2.5 | 2.5 | 2.5 |
| xylene sulfonate (93%) | 1.72 | — | — | — |
| lauryl ether sulfate 2EO (70%) | — | 9.7 | 9.7 | 7.1 |
| ammonium lauryl sulfate (25%) | — | — | — | 18.03 |
| alkyl benzene sulfonic acid | — | — | — | — |
| coco monoethanolamide | — | — | — | 1.0 |
| cocoamidopropyl betaine (30%) | — | 4.0 | 4.0 | — |
| salicylic acid | 0.4 | 0.3 | — | 0.3 |
| lactic acid (80%) | — | — | 2.5 | — |
| tartaric acid | 0.5 | — | — | — |
| citric acid | 0.5 | 0.55 | 0.55 | — |
| citric acid (50%) | — | *** | *** | 0.1 |
| sodium lactate | — | 0.4 | 0.4 | — |
| PCMX | — | 0.175 | 0.175 | — |
| Polyquaternium-7 | — | 0.1 | 0.1 | — |
| guar gum1 | — | — | — | 0.1 |
| glycerine | 1.0 | 0.9 | 0.9 | 1.0 |
| propylene glycol | 1.0 | 1.0 | 1.0 | 1.0 |
| sodium hydroxide (30%) | — | **** | **** | — |
| sodium hydroxide (50%) | — | — | 0.70 | 0.1 |
| sodium chloride (20%) | — | — | — | 8.0 |
| sodium chloride | — | * | * | — |
| sodium citrate dihydrate | — | 0.6 | 0.6 | — |
| sodium hydroxide (30%) | — | — | — | — |
| tetrasodium EDTA | 0.1 | 0.1 | 0.1 | 0.1 |
| cellulose thickener | — | 0.6 | 0.6 | — |
| preservative | — | 0.02 | 0.02 | 0.02 |
| fragrance1 | — | 0.3 | 0.3 | 0.3 |
| fragrance2 | — | — | — | 0.3 |
| menthol | — | — | — | 0.3 |
| opacifier | — | 1.5 | 1.5 | — |
| colorant | — | 0.015 | 0.015 | 0.0025 |
| di water | q.s. | q.s. | q.s. | q.s. |
| pH | 3.58 | 4.2-4.5 | 4.2-4.5 | — |
| Viscosity (cP) | — | 5000-8000 | 5000-8000 | — |

relative ratios: (wt/wt)

| | | | | |
|---|---|---|---|---|
| secondary alkane sulfonate | 4.5 | 4.0 | 4.0 | 4.0 |
| lauroyl sarcosinate | 1.88 | 0.21 | 0.21 | 0.21 |
| hydrotrope | 1.0 | 1.0 | 1.0 | 1.0 |

* sufficient anhydrous citric acid was added to provide a target pH
** sufficient sodium chloride (20%) was added to provide a target pH
*** sufficient citric acid (50%) was added to provide a target pH
**** sufficient sodium hydroxide (30%) was added to provide a target pH

TABLE 1B

| | E9 | E10 | E11 | E12 |
|---|---|---|---|---|
| secondary alkane sulfonate (60%) | 11.66 | 11.66 | 11.66 | 11.66 |
| lauroyl sarcosinate (30%) | — | — | — | — |
| cumene sulfonate (40%) | 7.5 | 5.0 | 2.5 | 1.25 |
| lauryl ether sulfate 2EO (70%) | — | — | — | — |
| ammonium lauryl sulfate (25%) | — | — | — | — |
| alkyl benzene sulfonic acid | — | — | — | — |
| coco monoethanolamide | — | — | — | — |
| cocoamidopropyl betaine (30%) | — | — | — | — |
| salicylic acid | 0.4 | 0.4 | 0.4 | 0.4 |
| lactic acid (80%) | — | — | — | — |
| tartaric acid | — | — | — | — |
| citric acid | — | — | — | — |
| citric acid soln. (50%) | *** | *** | *** | *** |
| sodium lactate | — | — | — | — |
| PCMX | — | — | — | — |
| Polyquaternium-7 | — | — | — | — |
| guar gum1 | — | — | — | — |
| glycerine | 1.0 | 1.0 | 1.0 | 1.0 |
| propylene glycol | 1.0 | 1.0 | 1.0 | 1.0 |
| sodium hydroxide (30%) | **** | **** | **** | **** |
| sodium hydroxide (50%) | — | — | — | — |
| sodium chloride (20%) | — | — | — | — |
| sodium chloride | — | — | — | — |
| sodium citrate dihydrate | — | — | — | — |
| sodium hydroxide (30%) | — | — | — | — |
| tetrasodium EDTA | 0.1 | 0.1 | 0.1 | 0.1 |
| cellulose thickener | — | — | — | — |
| preservative | — | — | — | — |
| fragrance1 | — | — | — | — |
| fragrance2 | — | — | — | — |
| menthol | — | — | — | — |
| opacifier | — | — | — | — |
| colorant | — | — | — | — |
| di water | q.s. | q.s. | q.s. | q.s. |
| pH | 4.0 | 4.0 | 4.0 | 4.0 |
| Viscosity (cP) | — | — | — | — |

relative ratios: (wt/wt)

| secondary alkane sulfonate | 2.33 | 3.5 | 7.0 | 13.99 |
|---|---|---|---|---|
| lauroyl sarcosinate | — | — | — | — |
| hydrotrope | 1.0 | 1.0 | 1.0 | 1.0 |
| | E13 | E14 | E15 | E16 |
| secondary alkane sulfonate (60%) | 10.0 | 8.33 | 6.6 | 5.0 |
| lauroyl sarcosinate (30%) | — | — | — | — |
| cumene sulfonate (40%) | 5 | 5 | 5 | 5 |
| lauryl ether sulfate 2EO (70%) | — | — | — | — |
| ammonium lauryl sulfate (25%) | — | — | — | — |
| alkyl benzene sulfonic acid | — | — | — | — |
| coco monoethanolamide | — | — | — | — |
| cocoamidopropyl betaine (30%) | — | — | — | — |
| salicylic acid | 0.4 | 0.4 | 0.4 | 0.4 |
| lactic acid (80%) | — | — | — | — |
| tartaric acid | — | — | — | — |
| citric acid | — | — | — | — |
| citric acid soln. (50%) | *** | *** | *** | *** |
| sodium lactate | — | — | — | — |
| PCMX | — | — | — | — |
| Polyquaternium-7 | — | — | — | — |
| guar gum1 | — | — | — | — |
| glycerine | 1.0 | 1.0 | 1.0 | 1.0 |
| propylene glycol | 1.0 | 1.0 | 1.0 | 1.0 |
| sodium hydroxide (30%) | **** | **** | **** | **** |
| sodium hydroxide (50%) | — | — | — | — |
| sodium chloride (20%) | — | — | — | — |
| sodium chloride | — | — | — | — |
| sodium citrate dihydrate | — | — | — | — |
| sodium hydroxide (30%) | — | — | — | — |
| tetrasodium EDTA | 0.1 | 0.1 | 0.1 | 0.1 |
| cellulose thickener | — | — | — | — |
| preservative | — | — | — | — |
| fragrance1 | — | — | — | — |
| fragrance2 | — | — | — | — |
| menthol | — | — | — | — |
| opacifier | — | — | — | — |
| colorant | — | — | — | — |
| di water | q.s. | q.s. | q.s. | q.s. |
| pH | 4.0 | 4.0 | 4.0 | 4.0 |
| Viscosity (cP) | — | — | — | — |
| relative ratios (wt/wt) | | | | |
| secondary alkane sulfonate | 3.0 | 2.5 | 2.0 | 1.5 |
| lauroyl sarcosinate | — | — | — | — |
| hydrotrope | 1.0 | 1.0 | 1.0 | 1.0 |

| | E17 | E18 |
|---|---|---|
| secondary alkane sulfonate (60%) | 3.33 | 1.66 |
| lauroyl sarcosinate (30%) | — | — |
| cumene sulfonate (40%) | 5 | 5 |
| lauryl ether sulfate 2EO (70%) | — | — |
| ammonium lauryl sulfate (25%) | — | — |
| alkyl benzene sulfonic acid | — | — |
| coco monoethanolamide | — | — |
| cocoamidopropyl betaine (30%) | — | — |
| salicylic acid | 0.4 | 0.4 |
| lactic acid (80%) | — | — |
| tartaric acid | — | — |
| citric acid | — | — |
| citric acid (50%) | *** | *** |
| sodium lactate | — | — |
| PCMX | — | — |
| Polyquaternium-7 | — | — |
| guar gum1 | — | — |
| glycerine | 1.0 | 1.0 |
| propylene glycol | 1.0 | 1.0 |
| sodium hydroxide (30%) | **** | **** |
| sodium hydroxide (50%) | — | — |
| sodium chloride (20%) | — | — |
| sodium chloride | — | — |
| sodium citrate dihydrate | — | — |
| sodium hydroxide (30%) | — | — |
| tetrasodium EDTA | 0.1 | 0.1 |
| cellulose thickener | — | — |
| preservative | — | — |
| fragrance1 | — | — |
| fragrance2 | — | — |
| menthol | — | — |
| opacifier | — | — |
| colorant | — | — |
| di water | q.s. | q.s. |
| pH | 4.0 | 4.0 |
| Viscosity (cP) | — | — |

relative ratios: (wt/wt)

| | | |
|---|---|---|
| secondary alkane sulfonate | 1.0 | 0.5 |
| lauroyl sarcosinate | — | — |
| hydrotrope | 1.0 | 1.0 |

*** sufficient citric acid (50%) was added to provide a target pH
**** sufficient sodium hydroxide (30%) was added to provide a target pH

TABLE 1C
(trigger spray)

| | E19 | E20 | E21 | E22 | E23 | E24 | E25 | E26 | E27 |
|---|---|---|---|---|---|---|---|---|---|
| lactic acid (80%) | 1.87 | 1.87 | 0.625 | 0.625 | 0.625 | 0.625 | 0.625 | 1.25 | 1.25 |
| citric acid | 0.5 | 0.5 | 0.5 | 0.5 | 0.5 | — | — | — | — |
| alkylpolyglycoside | 0.5 | 0.5 | 0.5 | 0.5 | 0.5 | 0.5 | 0.5 | 0.5 | 0.5 |
| (50%) | | | | | | | | | |
| dodecyl benzene | 2.5 | 2.5 | 3.75 | 2.5 | 2.5 | 3.75 | 3.75 | 3.75 | 3.75 |
| sulfonate (38%) | | | | | | | | | |
| dipropylene glycol n- | 2.0 | 2.0 | 2.0 | 2.0 | 2.0 | 2.0 | 2.0 | 2.0 | 2.0 |
| butyl ether | | | | | | | | | |
| cumene sulfonate | 2.5 | 2.5 | 2.5 | 2.5 | 2.5 | 2.5 | 2.5 | 2.5 | 2.5 |
| (40%) | | | | | | | | | |
| lauroyl sarcosinate | — | — | — | — | — | — | — | 1.429 | 1.429 |
| (35%) | | | | | | | | | |
| sodium hydroxide | q.s. | q.s. | q.s. | q.s. | q.s. | q.s. | q.s. | q.s. | q.s. |
| (30%) | | | | | | | | | |
| iminodisuccinate | — | 1.47 | — | — | — | — | — | — | — |
| (33%) | | | | | | | | | |
| DI water | q.s. | q.s. | q.s. | q.s. | q.s. | q.s. | q.s. | q.s. | q.s. |
| pH | 3.43 | 3.29 | 3.5 | 3.48 | 4.0 | 4.01 | 4.01 | 3.5 | 4.0 |

relative ratios (wt/wt)

| | | | | | | | | | |
|---|---|---|---|---|---|---|---|---|---|
| dodecyl benzene | 0.95 | 0.95 | 1.425 | 0.95 | 0.95 | 1.425 | 1.425 | 1.425 | 1.425 |
| sulfonate | | | | | | | | | |
| alkylpolyglycoside | 0.25 | 0.25 | 0.25 | 0.25 | 0.25 | 0.25 | 0.25 | 0.25 | 0.25 |
| cumene sulfonate | 1 | 1 | 1 | 1 | 1 | 1 | 1 | 1 | 1 |
| lauroyl sarcosinate | — | — | — | — | — | — | — | 0.50 | 0.50 |

antimicrobial testing: tested according to AOAC Official Method 961.02 -
Germicidal Spray Products as Disinfectants, results of viable test slide/
total test slides (at 100%, and/or at 50% aqueous dilution)

| | | | | | | | | | |
|---|---|---|---|---|---|---|---|---|---|
| S. aureus | 0/20 (at | 0/20 (at | 0/20 (at | 8/52 (at | 1/59 (at | 7/60 (at | 0/30 (at | 0/60 (at | 1/60 (at |
| | 100%, | 100%, | 100%, | 50%, | 100%, | 100%, | 100%, | 100%, | 100%, |
| | 5 min.); | 5 min.); | 5 min.); | 5 min) | 5 min.) | 5 min.) | 5 min.) | 5 min.) | 5 min.) |
| | 0/20 at | 0/20 at | | | | | | | |
| | (50%, | (50%, | | | | | | | |
| | 5 min.) | 5 min.) | | | | | | | |
| S. enterica | 0/60 (at | 0/60 (at | — | 0/60 (at | 0/60 (at | — | — | — | — |
| | 50%, | 50%, | | 50%, | 50%, | | | | |
| | 5 min) | 5 min) | | 5 min) | 5 min) | | | | |

(wipes)

| | E28 | E29 | E30 | E31 |
|---|---|---|---|---|
| lactic acid (80%) | 1.87 | 1.87 | 1.87 | 1.87 |
| citric acid | 1.5 | 1.5 | 1.5 | 1.5 |
| alkylpolyglycoside (50%) | 0.6 | 0.6 | 0.6 | 0.6 |
| dodecyl benzene sulfonate (38%) | 1.25 | 1.25 | 0 | 0 |
| dipropylene glycol n-butyl ether | 1.0 | 1.0 | 1.0 | 1.0 |
| cumene sulfonate (40%) | 1.25 | 1.25 | 1.25 | 1.25 |
| secondary alkane sulfonate (30%) | 1.66 | 1.66 | 3.33 | 3.33 |
| 2-ethylhexanol propoxylated | 0 | 0 | 0 | 0 |
| ethoxylated polymer | | | | |
| sodium hydroxide | q.s. | q.s. | q.s. | q.s. |
| DI water | q.s. | q.s. | q.s. | q.s. |
| pH | 3.8 | 3.8 | 3.43 | 3.43 |

substrate; loading ratio (wt/wt)

| | | | | |
|---|---|---|---|---|
| Wipe01 | 4:1 | — | 4:1 | — |
| Wipe02 | — | 4:1 | — | 4:1 |

relative ratios (wt/wt)

| | | | | |
|---|---|---|---|---|
| dodecyl benzene sulfonate + | 2 | 2 | 2 | 2 |
| secondary alkane sulfonate | | | | |
| alkylpolyglycoside | 0.6 | 0.6 | 0.6 | 0.6 |
| cumene sulfonate | 1 | 1 | 1 | 1 |

antimicrobial testing: AOAC Official Method 961.02 - Germicial Spray
Products as Disinfectants (4 min contact time), moofied for towelettes

| | | | | |
|---|---|---|---|---|
| S. aureus | 0/20 | 2/20 | 5/20 | 13/20 |
| S. enterica | 1/20 | — | — | — |

Wipe01 - a nonwoven wipe substrate, 38% polypropylene, 62% wood pulp; dry weight: 45 g/m²
Wipe02 - a nonwoven wipe substrate, 100% polyethylene terephtalate; dry weight: 50 g/m²

TABLE 1D

| | E32 | E33 | E34 | E35 | E36 |
|---|---|---|---|---|---|
| dodecylbenzene | 2.0 | 4.0 | 4.0 | 2.0 | 2.0 |
| sulfonate (38%) | | | | | |
| xylene sulfonate (40%) | 1.25 | 1.25 | — | — | — |
| cumene sulfonate (40%) | — | — | 0.6 | 0.6 | 0.6 |
| alcohol ethoxylate | — | — | 0.25 | 0.25 | — |
| alkylpolyglycoside | 0.5 | 0.4 | — | — | — |
| citric acid | — | 3 | — | — | — |
| lactic acid (80%) | 2.5 | — | 3.75 | 2.5 | 2.5 |
| trisodium citrate | 0.45 | 0.45 | — | — | — |
| dipropylene glycol n- | — | — | 0.5 | 0.5 | — |
| butyl ether | | | | | |
| fragrance | — | — | 0.05 | 0.05 | 0.05 |
| di water | q.s. | q.s. | q.s. | q.s. | q.s. |
| pH | 3.01 | 3.01 | 2.3 | 2.3 | 2.3 |

relative ratios (wt/wt) (a):(b):

| | | | | | |
|---|---|---|---|---|---|
| (a) dodecylbenzene | 1.52:1 | 1.52:1 | 3.17:1 | 1.58:1 | 1.58:1 |
| sulfonate:(b) | | | | | |
| xylene sulfonate + | | | | | |
| cumene sulfonate | | | | | |
| wipe and loading | 4:1 | 4:1 | 4:1 | 4:1 | 4:1 |
| (% wt/% wt) | | | | | |

Antimicrobial efficacy (Log₁₀) - wipe-loading

| | | | | | |
|---|---|---|---|---|---|
| S. aureus - Wipe03 | 0/20 | 1/20 | 0/60 | 1/60 | 4/60 |
| S. enterica - Wipe03 | 0/20 | 0/20 | 0/60 | 0/60 | 1/60 |
| S. aureus - Wipe04 | 3/20 | — | — | — | — |
| S. enterica - Wipe04 | 1/20 | — | — | — | — |
| S. aureus - Wipe01 | 3/20 | — | — | — | — |
| S. enterica - Wipe01 | 0/20 | — | — | — | — |

| | E37 | E38 | E39 | E40 | E41 | E42 |
|---|---|---|---|---|---|---|
| dodecylbenzene sulfonate | 0.105 | 0.105 | 0.105 | 0.105 | 0.105 | 0.105 |
| (38%) | | | | | | |
| lauryl sulfate (98%) | — | — | — | — | — | 1.0 |
| lauryl sulfate (30%) | 5.0 | 3.33 | 2.5 | 3.33 | 3.33 | — |
| primary alkane sulfonate | — | — | — | — | — | 0.08 |
| (98%) | | | | | | |
| xylene sulfonate (40%) | 0.124 | 0.124 | 0.124 | 0.124 | 0.124 | 0.124 |
| cumene sulfonate (40%) | — | — | — | — | — | — |
| linear alcohol ethoxylate | — | — | — | — | — | — |
| branched alcohol | 0.25 | 0.25 | 0.25 | 0.25 | 0.25 | — |
| ethoxylate | | | | | | |
| alkylpolyglucoside (50%) | — | — | — | — | — | 1.0 |
| alkylpolyglucoside (63%) | 0.4 | 0.4 | 0.4 | 0.4 | 0.4 | — |
| citric acid | — | — | — | — | — | — |
| sodium citrate dihydrate | — | — | — | — | — | 0.457 |
| lactic acid (80%) | 3.8 | 2.5 | 2.5 | 2.5 | 3.125 | 2.5 |
| trisodium citrate | — | — | — | — | — | — |
| diethylene glycol | 1.0 | 1.0 | 1.0 | — | — | — |
| monoethyl ether | | | | | | |
| ethanol | — | — | — | 0.4 | 0.4 | — |
| fragrance | — | — | — | 0.05 | 0.05 | — |
| di water | q.s. | q.s. | q.s. | q.s. | q.s. | q.s. |
| pH | 2.3 | 2.3 | 2.3 | 2.3 | 2.3 | 3.02 |

relative ratios (wt): (a):(b):(c):(d)

| | | | | | | |
|---|---|---|---|---|---|---|
| (a) dodecylbenzene | 0.8:1:30.21:5 | 0.8:1:20.16:5 | 0.8:1:16.12:5 | 0.8:1:22.4:5 | 0.8:1:22.4:5 | 0.8:1:21.33:10 |
| sulfonate:(b) | | | | | | |
| xylene sulfonate + | | | | | | |
| cumene sulfonate:(c) | | | | | | |
| lauryl sulfate + primary | | | | | | |
| alkane sulfonate | | | | | | |
| (d) alkylpolyglucoside | | | | | | |

wipe and loading (wt/wt)

| | | | | | | |
|---|---|---|---|---|---|---|
| Wipe03 | 4.5:1 | 4.5:1 | 4.5:1 | — | — | — |
| Wipe04 | — | — | — | 4.5:1 | 4.5:1 | — |

Antimicrobial efficacy (Log₁₀)

| | | | | | | |
|---|---|---|---|---|---|---|
| S. aureus - Wipe03 | 0/60 | 0/60 | 3/60 | — | — | — |
| S. enterica - Wipe03 | — | — | — | — | — | — |

| | E43 |
|---|---|
| dodecylbenzene sulfonate | 0.105 |
| (38%) | |
| lauryl sulfate (98%) | 1.0 |
| lauryl sulfate (30%) | — |
| primary alkane sulfonate | 0.08 |
| (98%) | |
| xylene sulfonate (40%) | 0.124 |
| cumene sulfonate (40%) | — |
| linear alcohol ethoxylate | — |
| branched alcohol ethoxylate | — |
| alkylpolyglucoside (50%) | 1.0 |
| alkylpolyglucoside (63%) | — |
| citric acid | — |
| sodium citrate dihydrate | 0.457 |
| lactic acid (80%) | 2.5 |
| trisodium citrate | — |
| diethylene glycol monoethyl | — |
| ether | |
| ethanol | — |
| fragrance | — |
| di water | q.s. |
| pH | 3.02 |

relative ratios (wt/wt): (a):(b):(c):(d)

| | |
|---|---|
| (a) dodecylbenzene | 0.8:1:21.33:10 |
| sulfonate:(b) | |
| xylene sulfonate + | |
| cumene sulfonate:(c) | |
| lauryl sulfate + primary | |
| alkane sulfonate | |
| (d) alkylpolyglucoside | |

Antimicrobial efficacy (Log₁₀)⁺⁺

| | |
|---|---|
| S. aureus | 0/20 |
| S. enterica | 1/60, 0/60 |

Wipe01 - a nonwoven wipe substrate, 38% polypropylene, 62% wood pulp; dry weight: 45 g/m²
Wipe03 - a nonwoven wipe substrate, blend of polyethylene terephtalate, polypropylene and viscose fibers; dry weight: 45 g/m²
Wipe04 - a nonwoven wipe substrate, Lyocell (rayon) fibers; dry weight 45 g/m²
++ the composition was tested without loading onto a dry carrier substrate, according to the AOAC Germicidal Spray Test, for a 10 minute contact time

TABLE 1E

| | E44 | E45 |
|---|---|---|
| secondary alkane sulfonate (60%) | 7.2 | 7.2 |
| lauroyl sarcosinate (30%) | 3.0 | 3.0 |
| cumene sulfonate (40%) | 1.6 | 1.6 |
| salicylic acid | — | — |
| lactic acid (80%) | — | — |
| sodium lactate | — | — |
| tartaric acid | — | — |
| citric acid | — | — |
| citric acid soln. (50%) | *** | *** |
| sodium hydroxide soln. (30%) | **** | **** |
| di water | q.s. | q.s. |
| pH | 4.0 | 3.5 |
| Viscosity (cP) | — | — |
| Ratios: (wt/wt) | — | — |
| secondary alkane sulfonate | 6.75 | 6.75 |
| lauroyl sarcosinate | 1.41 | 1.41 |
| hydrotrope | 1.0 | 1.0 |

*** sufficient citric acid soln. (50%) was added to provide a target pH
**** sufficient sodium hydroxide soln. (30%) was added to provide a target pH

TABLE C

| | C1 | C2 | C3 |
|---|---|---|---|
| secondary alkane sulfonate (60%) | — | — | — |
| lauroyl sarcosinate (30%) | — | — | — |
| cumene sulfonate (40%) | — | — | — |
| lauryl ether sulfate 2EO (70%) | 15.6 | 8.57 | 7.1 |
| ammonium lauryl sulfate (25%) | — | — | 25 |
| alkyl benzene sulfonic acid | — | — | — |
| coco monoethanolamide | — | 0.8 | 1.0 |
| cocoamidopropyl betaine | 4 | 6.9 | — |
| sodium coco-sulfate | — | 4 | — |
| salicylic acid | 0.3 | 0.4 | 0.3 |
| PCMX | 0.175 | — | — |
| lactic acid (90%) | — | — | — |
| sodium lactate | 0.4 | — | — |
| Polyquaternium-7 | 0.1 | 0.4 | — |
| guar gum1 | — | — | 0.1 |
| guar gum2 | — | 0.4 | — |
| glycerine | 0.9 | 5 | 1.0 |
| propylene glycol | 1 | — | 1.0 |
| PEG-8 | — | 2.5 | — |
| sodium hydroxide soln. (50%) | — | — | — |
| sodium hydroxide soln. (30%) | 0.133 | 0.1 | — |
| sodium chloride | 0.55 | 0.8 | — |
| sodium chloride soln. (20%) | — | — | — |
| tartaric acid | — | — | — |
| citric acid | 0.55 | 0.3 | 0.1 |
| citric acid soln. (50%) | — | — | — |
| sodium citrate dihydrate | 0.6 | 0.3 | — |
| sodium hydroxide (30%) | — | — | — |
| tetrasodium EDTA | 0.1 | 0.2 | 0.1 |
| cellulose thickener | 0.6 | — | — |
| preservative | 0.02 | 0.02 | 0.02 |
| fragrance1 | 0.3 | — | 0.3 |
| fragrance2 | 0.48 | 0.3 | 0.3 |
| opacifier | — | — | — |
| glycol distearate | — | 1 | — |
| colorant | 0.00072 | — | 0.0025 |
| di water | q.s. | q.s. | q.s. |
| pH | — | — | — |

| | C4 |
|---|---|
| secondary alkane sulfonate (60%) | — |
| lauroyl sarcosinate (30%) | 0.7 |
| cumene sulfonate (40%) | 2.5 |
| lauryl ether sulfate 2EO (70%) | 7.1 |
| ammonium lauryl sulfate (25%) | 14.7 |
| alkyl benzene sulfonic acid1 | 10.0 |
| coco monoethanolamide | 1.0 |
| cocoamidopropyl betaine | — |
| sodium coco-sulfate | — |
| salicylic acid | — |
| PCMX | — |
| lactic acid (90%) | 2.5 |
| sodium lactate | — |
| Polyquaternium-7 | — |
| guar gum1 | 0.1 |
| guar gum2 | — |
| glycerine | 1.0 |
| propylene glycol | 1.0 |
| PEG-8 | — |
| sodium hydroxide soln. (50%) | 0.1 |
| sodium hydroxide soln. (30%) | — |
| sodium chloride | — |
| sodium chloride soln. (20%) | — |
| tartaric acid | — |
| citric acid | — |
| citric acid soln. (50%) | 0.1 |
| sodium citrate dihydrate | — |
| sodium hydroxide (30%) | — |
| tetrasodium EDTA | 0.1 |
| cellulose thickener | — |
| preservative | 0.02 |
| fragrance1 | — |
| fragrance2 | — |
| opacifier | — |
| glycol distearate | — |
| colorant | — |
| di water | q.s. |
| pH | 4.19 |
| Viscosity | 4000-8000 |

* sufficient sodium chloride (100%) was added to provide a target viscosity
** sufficient sodium chloride soln. (20%) was added to provide a target viscosity
*** sufficient citric acid soln. (50%) was added to provide a target pH
**** sufficient sodium hydroxide soln. (30%) was added to provide a target pH

The identity of the constituents used, their source material (tradename and/or supplier), and their % wt. actives “as supplied” are indicated on Table 2, following:

TABLE 2

| | |
|---|---|
| secondary alkane sulfonate (60%) | secondary alkane sulfonate, sodium salt, supplied as |
| | Hostapur SAS-60, 60% wt. actives (ex. Nease Co.), |
| secondary alkane sulfonate (30%) | secondary alkane sulfonate, sodium salt, supplied as |
| | Hostapur SAS-30, 30% wt. actives (ex. Nease Co.), |
| dodecyl benzene sulfonate (38%) | dodecyl benzene sulfonate, sodium salt, supplied in |
| | Biosoft D40 (ex. Stepan Co.) |
| lauroyl sarcosinate (30%) | sodium lauroyl sarcosinate, supplied as Crodasinic LS- |
| | 30, 30% wt. actives (ex. Croda), |
| cumene sulfonate (40%) | sodium cumene sulfonate, supplied as Naxonate 40SC, |
| | 40% wt. actives (ex. Nease Co.), |
| xylene sulfonate (93%) | sodium xylene sulfonate, (93% wt. actives) |
| xylene sulfonate (40%) | sodium xylene sulfonate, (40% wt. actives) supplied as |
| | Stepanate SXS (ex. Stepan Co.) |
| lauryl sulfate (98%) | lauryl sulfate, sodium salt, (98% wt. actives) supplied as |
| | Stepanol WA-100NF/USP (ex. Stepan Co.) |
| lauryl sulfate (30%) | lauryl sulfate, sodium salt, (30% wt. actives) supplied as |
| | Stepanol WA-Extra PCK (ex. Stepan Co.) |
| primary alkane sulfonate (38%) | alkane sulfonate, sodium salt (38% wt.) supplied as |
| | BioTerge PAS-86 (ex. Stepan Co.) |
| lauryl ether sulfate 2EO (70%) | sodium lauryl ether sulfate 2EO, supplied as Galaxy LES |
| | 70, 70% wt. actives (ex. Galaxy Chem. Co.), or Texapon |
| | N 70, 70% wt. actives (ex. BASF) |
| ammonium lauryl sulfate (25%) | supplied as Rhodia ALS, 25% wt. actives, (ex. Rhodia), |
| alkyl benzene sulfonic acid | supplied as Biosoft D-40, 38%% wt. actives, (ex. Stepan |
| | Co.) |
| sodium coco-sulfate | supplied as Mackol CAS-100N, 90-100% wt. actives (ex. |
| | Rhodia) |
| 2-ethylhexanol propoxylated | 2-ethylhexanol propoxylated ethoxylated polymer, |
| ethoxylated polymer | nonionic surfactant, supplied in EcoSurf (ex. DOW |
| | Chem.) |
| fatty alcohol polyglycol ether | Laureth-7, supplied in Genapol LA 070S (ex. Clariant) |
| alkylpolyglycoside | caprylyl/myristyl glucoside, supplied in Glucopon 425N |
| | (ex. BASF) |
| alkylpolyglucoside (50%) | caprylyl/myristyl glucoside, (50% wt. actives) supplied as |
| | Glucopon 425N (ex. BASF) |
| alkylpolyglucoside (63%) | caprylyl/decyl glucoside, (63% wt. actives) supplied as |
| | Glucopon 215UP (ex. BASF) |
| branched alcohol ethoxylate | nonionic surfactant, ethyl hexanol based ethoxylated, |
| | (100% wt. actives) supplied as Ecosurf EH6 (ex. DOW |
| | Chem Co.) |
| linear alcohol ethoxylate | C₉-C₁₁ linear primary alcohol ethoxylate (6EO) nonionic |
| | surfactant (100% wt. actives), supplied as Tomadol 91-6 |
| | (ex. ) |
| coco monoethanolamide | supplied as Galaxy 100, 100% wt. actives (ex. Galaxy |
| | Chem. Co.) or as Macamide CMS, 100% wt. actives (ex. |
| | Rhodia) |
| cocoamidopropyl betaine (30%) | supplied as Empigen BS/FA, 100% wt. actives (ex. |
| | Huntsman) |
| salicylic acid | laboratory grade salicylic acid, 100% wt. active (ex. |
| | Aldrich, Sigma or other supplier) |
| lactic acid (90%) | laboratory grade salicylic acid, 90% wt. active (ex. |
| | Aldrich, Sigma or other supplier) |
| lactic acid (80%) | laboratory grade salicylic acid, 80% wt. active (ex. |
| | Aldrich, Sigma or other supplier) |
| sodium lactate | sodium lactate, 100% wt. actives (ex. Purac) |
| sodium hydroxide (30%) | aqueous solution of in di water of laboratory grade |
| | sodium hydroxide, 30% wt. actives, (ex. Aldrich, Sigma or |
| | other supplier) |
| sodium hydroxide (50%) | aqueous solution of in di water of laboratory grade |
| | sodium hydroxide, 50% wt. actives, (ex. Aldrich, Sigma or |
| | other supplier) |
| sodium chloride | laboratory grade sodium chloride, 100% wt. active (ex. |
| | Aldrich, Sigma or other supplier) |
| sodium chloride (20%) | aqueous solution of in di water of laboratory grade |
| | sodium chloride, 20% wt. actives, (ex. Aldrich, Sigma or |
| | other supplier) |
| tartaric acid | anhydrous tartaric acid, laboratory grade, 100% wt. |
| | actives (ex. Aldrich, Sigma or other supplier) |
| citric acid | anhydrous citric acid, laboratory grade, 100% wt. actives |
| | (ex. Aldrich, Sigma or other supplier) |
| citric acid soln. (50%) | aqueous solution in di water of laboratory grade citric |
| | acid, 50% wt. actives, (ex. Aldrich, Sigma or other |
| | supplier) |
| sodium citrate dihydrate | trisodium citrate dehydrate, 100% wt. (ex. Aldrich, Sigma |
| | or other supplier) |
| trisodium citrate | anhydrous trisodium citrate (100% wt. actives) laboratory |
| | grade |
| guar gum1 | supplied as Jaguar C-162, guar |
| | hydroxyproplyletrimonium chloride, 100% wt. actives (ex. |
| | Rhodia) |
| guar gum2 | supplied as Jaguar C-17, guar hydroxyproplyletrimonium |
| | chloride, 100% wt. actives (ex. Rhodia) |
| cellulose thickener | hydroxypropyl methylcellulose, supplied as Methocel 856 |
| | N PC grade, 100% wt. actives (ex. DOW Chem. Co.) |
| glycerine | laboratory grade glycerine, 100% wt. active (ex. Aldrich, |
| | Sigma or other supplier) |
| propylene glycol | laboratory grade propylene glycol, 100% wt. active (ex. |
| | DOW Chem, Aldrich, or other supplier) |
| dipropylene glycol n-butyl ether | dipropylene glycol n-butyl ether, supplied as Dowanol |
| | DPnB (ex. DOW) |
| diethylene glycol monoethyl ether | diethylene glycol monoethyl ether, (99-100% wt. actives) |
| | supplied as Carbitol (ex. DOW) |
| ethanol | ethanol (95-100% wt. actives) laboratory grade |
| acrylate polymer | sodium acrylate polymer, supplied in Syntran 4022 (ex. |
| | Interpolymer) |
| PEG-8 | Polyethylene glycol 8, 100% wt. active, supplied as |
| | Puracare E400 NF (ex. BASF), or Carbowax PEG 400 |
| | NF (ex. DOW) |
| glycol distearate | supplied as Alkamuls EGDS 515, 58-65% wt. actives (ex. |
| | Solvay) |
| tetrasodium EDTA | Trilon B SP (ex. BASF) |
| iminodisuccinate (33%) | tetrasodium iminodisuccinate, supplied as Baypure |
| | CX100 (ex. Lanxess) |
| PCMX | para-chloro-meta-xylenol, 100% wt. actives |
| Polyquaternium-7 | Polyquaternium-7, copolymer of acrylamide and |
| | diallyldimethylammonium chloride, % wt. actives (ex. |
| | Rhodia) |
| preservative | Kathon CG (ex. Rohm & Haas/DOW Chem. Co.) |
| fragrance1 | menthol crystals, proprietary composition of its supplier |
| fragrance2 | fragrance, proprietary composition of its supplier |
| menthol | used as supplied |
| opacifier | supplied as Empipearl XA/400X, 38% wt. actives, (ex. |
| | Huntsman) |
| colorant | D&C Blue No. 1 (1% aqueous solution) |
| di water | deionized water |

The following Tables 3A and 3B report the antimicrobial efficacy of certain example formulations previously disclosed.

The test protocol of Table 3A was that described in European Standard EN 1276:2009—Chemical Disinfectants and Antiseptics—Quantitative suspension test for the evaluation of bactericidal activity of chemical disinfectants used in the food, industrial, domestic and institutional areas. Where indicated on the table, the test was performed under “dirty” conditions (standardized horse serum was added to the test culture to achieve a final concentration of 5%). The log₁₀ reduction of the compositions are indicated on Table 3A.

TABLE 3A

| | contact | | | | | | |
|---|---|---|---|---|---|---|---|
| | time | concentration | conditions | S. aureus | E. coli | P. aeruginosa | E. hirae |
| E2 | 1 min. | 80% | dirty | 3.84 | 4.23 | >5.25 | 3.04 |
| E3 | 1 min. | 80% | dirty | >5.34 | 4.47 | >5.25 | >5.47 |
| E1 | 1 min. | 80% | dirty | >5.14 | >5.58 | >5.04 | >5.55 |
| E5 | 1 min. | 80% | dirty | >5.14 | >5.58 | >5.04 | >5.55 |
| E44 | — | — | — | >5.44 | >5.44 | — | — |
| E45 | — | — | — | >5.44 | >5.44 | — | — |
| E4 | 5 min. | 80% | dirty | >4.72 | 4.77 | 5.36 | >5.51 |
| E6 | 5 min. | 80% | dirty | 4.78 | 4.84 | >5.68 | >6.02 |
| E6 | 1 min. | 80% | dirty | 3.59 | 1.29 | 5.56 | 4.94 |
| E7 | 5 min. | 80% | dirty | 3.34 | 3.35 | >5.68 | 3.84 |
| E8 | 1 min. | 80% | dirty | 3.86 | 0.61 | 1.88 | >5.72 |
| E8 | 5 min | 80% | dirty | >4.72 | 4.57 | 5.03 | >5.51 |

The above reported results demonstrate excellent antimicrobial efficacy of the indicated Example compositions.

Certain of the example compositions, were tested as liquids or spray products, viz., without first being applied to a carrier substrate were evaluated according to the protocols of the AOAC Official Method 961.02 Germicidal Spray Products against one or more challenge microorganisms. As is appreciated by the skilled practitioner in the art, the results of the said modified AOAC Germicidal Spray Test indicates the number of test slides wherein the tested organism remains viable after contact for 1, 4, 5 or 10 minutes with a tested composition/total number of tested substrates evaluated in accordance with the modified AOAC Germicidal Spray Test. Thus, a result of “0/20” indicates that of 20 test slides bearing the test organism and contacted for the contact time (minutes) in a tested composition, 0 test slides had viable (live) test organisms at the conclusion of the test. Similarly a result of “1/60” indicates that of 60 test slides bearing the test organism and contacted for the contact time (minutes) in a tested composition, 1 test substrate had viable (live) test organisms at the conclusion of the test. Both are indicative of a highly antimicrobially effective tested composition. The reported results indicate the number of test cylinders with live test organisms/number of test cylinders tested for each example formulation and organism tested. Certain of these results, and the testing conditions are disclosed with reference to one or more of foregoing Tables 1 through 5.

Other of the example compositions were first applied to a carrier (wipe) at the indicated loading levels before being were evaluated according to the protocols of the AOAC Official Method 961.02 Germicidal Spray Products as Disinfectants, which is modified for use with towelettes according to EPA Series 810 guidelines (OCSPP 810.2200: Disinfectants for Use on Hard Surfaces—Efficacy Data Recommendations), against one or more challenge microorganisms. The test was performed under “dirty” conditions (standardized horse serum was added to the test culture to achieve a final concentration of 5%). The testing was performed for a 4 minute contact time. As is appreciated by the skilled practitioner in the art, the results of the said modified AOAC Germicidal Spray Test indicates the number of test slides wherein the tested organism remains viable after contact for the indicated minutes with a tested composition/total number of tested substrates (slides) evaluated in accordance with the modified AOAC Germicidal Spray Test. The reported results indicate the number of test slides with live test organisms/number of test slides tested for each example formulation and organism tested. The reported results indicate the number of test slides with live test organisms/number of test slides tested for each example formulation and organism tested. Certain of these results, and the testing conditions are disclosed with reference to one or more of foregoing Tables 1 through 5.

Table C3A lists the antimicrobial efficacy of certain comparative formulations, which were also tested using European Standard EN 1276:2009—Chemical Disinfectants and Antiseptics—Quantitative suspension test for the evaluation of bactericidal activity of chemical disinfectants used in the food, industrial, domestic and institutional areas.

TABLE C3A

| | contact | | | | | | |
|---|---|---|---|---|---|---|---|
| | time | concentration | conditions | S. aureus | E. coli | P. aeruginosa | E. hirae |
| C1 | 1 min. | 80% | dirty | 3.55 | 3.41 | 3.78 | 4.37 |
| C2 | 1 min. | 80% | dirty | 2.01 | 0.65 | 1.59 | 3.58 |
| C3 | 1 min. | 80% | dirty | 2.5 | 0.53 | 0.63 | >5.72 |

1. An acidic aqueous treatment composition having a pH not in excess of 4.5 which necessarily comprises:
as an antimicrobial constituent, lactic acid, citric acid, substituted lactic acid, substituted citric acid, tartaric acid, substituted tartaric acid, derivatives thereof and/or salts thereof; and,
an antimicrobially enhancing effective amount of a ternary anionic surfactant system which necessarily comprises one or more of each of:
(a) a secondary alkane sulfonate surfactant compound(s),
(b) an N-acyl sarcosinate compound and
(c) an anionic hydrotrope surfactant compound(s), wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and wherein the ternary anionic surfactant system boosts the antimicrobial efficacy of the antimicrobial constituent present as compared to like compositions wherein (a), (b) and (c) are omitted.

2. An acidic aqueous treatment composition having a pH not in excess of 4.5 which treatment composition necessarily comprises:
as an antimicrobial constituent, lactic acid, citric acid, substituted lactic acid, substituted citric acid, tartaric acid, substituted tartaric acid, derivatives thereof and/or salts thereof; and,
an antimicrobially enhancing effective amount of a binary anionic surfactant system which necessarily comprises one or more of each of:
(a) a secondary alkane sulfonate surfactant compound(s), and
(c) an anionic hydrotrope surfactant compound(s), and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and wherein the binary anionic surfactant system boosts the antimicrobial efficacy of the antimicrobial constituent present as compared to like compositions wherein (a) and (c) are omitted.

3. An acidic aqueous treatment composition having a pH not in excess of 4.5, which composition is particularly adapted for the treatment of hard and/or soft surfaces which have been contacted by a non-human animal, which treatment composition necessarily comprises:
as an antimicrobial constituent, lactic acid, citric acid, substituted lactic acid, substituted citric acid, tartaric acid, substituted tartaric acid, derivatives thereof and/or salts thereof; and,
a ternary surfactant constituent system which necessarily comprises one or more of each of:
(a) one or more alkyl sulfonate surfactant compound(s) selected from alkylbenzene sulfonate compound(s) and a secondary alkane sulfonate surfactant compound(s);
(b) one or more alkylglycoside nonionic surfactant compound(s); and;
(c) an aromatic hydrotrope compound(s), and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and wherein the ternary surfactant constituent system boosts the antimicrobial efficacy of the antimicrobial constituent present as compared to a like composition wherein (a), (b) and (c) are omitted.

4. An acidic aqueous treatment composition having a pH not in excess of 4.5, which composition is particularly adapted for the treatment of hard and/or soft surfaces which have been contacted by a non-human animal, e.g. canines, felines, reptiles which are typically considered household pets, which treatment composition necessarily comprises at least:
as an antimicrobial constituent, one or more organic acids selected from lactic acid, citric acid, substituted lactic acid, substituted citric acid, tartaric acid, substituted tartaric acid, derivatives thereof and/or salts thereof; and,
a quaternary surfactant constituent system which necessarily comprises one or more of each of:
(a) an alkyl sulfonate surfactant compound(s) selected from an alkylbenzene sulfonate compound and a secondary alkane sulfonate surfactant compound(s);
(b) one or more alkylglycoside nonionic surfactant compound(s);
(c) an aromatic hydrotrope compound(s); and,
(d) an N-acyl sarcosinate compound(s),
and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and wherein the quaternary constituent system boosts the antimicrobial efficacy of the disinfecting constituent present as compared to a like composition wherein (a), (b), (c) and (d) are omitted.

5. An acidic, largely aqueous hard surface treatment composition which features low toxicity to humans and animals, and which also provides a good antimicrobial effect against undesired microorganisms, which is optionally but preferably provided with a carrier substrate, which hard surface treatment composition necessarily comprises:
lactic acid, citric acid, substituted lactic acid, substituted citric acid, tartaric acid, substituted tartaric acid, derivatives thereof and/or salts thereof; and,
a binary system of anionic compounds which necessarily includes:
(a) one or more alkylbenzene sulfonate surfactant compounds; and
(b) an anionic aromatic hydrotrope compound which includes an aryl moiety and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and preferably the binary system boosts the antimicrobial efficacy of the disinfecting constituent present as compared to a like composition wherein (a) and (b) are omitted; and further wherein the treatment composition exhibits a pH of from about 1 to 4.5.

6. An acidic, largely aqueous hard surface treatment composition which features low toxicity to humans and animals, but which provides a good antimicrobial effect against undesired microorganisms, which is optionally but preferably supplied with a carrier substrate, which hard surface treatment composition necessarily comprises:
as an antimicrobial constituent, lactic acid, citric acid, substituted lactic acid, substituted citric acid, tartaric acid, substituted tartaric acid, derivatives thereof and/or salts thereof; and,
a ternary system of surfactant compounds which necessarily includes:
(a) one or more alkylbenzene sulfonate surfactant compounds; and
(b) one or more anionic aromatic hydrotrope compounds includes an aryl moiety, and
(c) one or more further anionic surfactant(s) other than (a) and (b) which further anionic surfactant includes at least one surfactant selected from linear alkane sulfonate and/or a linear alkane sulfate, and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and wherein the ternary system boosts the antimicrobial efficacy of the disinfecting constituent present as compared to a like composition wherein (a), (b) and (c) are omitted; and further preferably, wherein the treatment composition exhibits a pH of from about 1 to 4.5.

7. An acidic, largely aqueous hard surface treatment composition which features low toxicity to humans and animals, but which provides a good antimicrobial effect against undesired microorganisms, which is optionally but preferably supplied with a carrier substrate, which hard surface treatment composition necessarily comprises:
as an antimicrobial constituent, lactic acid, citric acid, substituted lactic acid, substituted citric acid, tartaric acid, substituted tartaric acid, derivatives thereof and/or salts thereof; and,
a quaternary system of surfactant compounds which necessarily includes:
(a) one or more alkylbenzene sulfonate surfactant compounds; and
(b) one or more anionic aromatic hydrotrope compounds which includes an aryl moiety,
(c) one or more further anionic surfactant(s) other than (a) and (b) which further anionic surfactant includes at least one surfactant selected from linear alkane sulfonate and/or a linear alkane sulfate, and
(d) one or more alkylglucoside nonionic surfactant compounds,
and wherein the treatment composition provides an antimicrobial benefit against one or more undesired pathogens, preferably one or more of bacteria selected from the genus: Salmonella, especially S. enterica, and/or of the genus Staphylococcus, especially Staphylococcus aureus and/or of the genus Eserichia, especially E. coli, and/or of the genus Enterococcus, and especially E. hirae, and preferably the quaternary system boosts the antimicrobial efficacy of the disinfecting constituent present as compared to a like composition wherein (a), (b), (c) and (d) are omitted; and further preferably, the treatment composition exhibits a pH of from about 1 to 4.5.

8. An acidic, largely aqueous hard surface treatment composition having a pH of about 4.5 or less and in which an antimicrobial constituent selected from salicylic acid and lactic acid is omitted, but citric acid is present concurrently with a ternary anionic surfactant system is present and which system necessarily comprises one or more of each of: (a) a secondary alkane sulfonate surfactant compound(s), (b) an N-acyl sarcosinate compound(s) and (c) an anionic hydrotrope surfactant compound(s) and which (a), (b) and (c) provide an effective antimicrobial benefit against undesired microorganisms, preferably one or more of the bacteria selected from the genus: Salmonella, especially Salmonella enterica, and/or the genus Staphylococcus, especially Staphylococcus aureus.

9. A treatment composition according to claim 1, wherein the composition exhibits an antimicrobial effect (preferably sanitizing or disinfecting) and preferably a broad spectrum antimicrobial effect, as demonstrated according to one or more of the following known test protocols: (i) AOAC Official Method 961.02 Germicidal Spray Products as Disinfectants, including also when modified for use with towelettes according to EPA Series 810 guidelines (OCSPP 810.2200: Disinfectants for Use on Hard Surfaces—Efficacy Data Recommendations), (ii) AOAC Official Method 955.14, 955.15 (preferably against S. aureus and/or S. enterica); (iii) European Standard EN 1276:2009—Chemical Disinfectants and Antiseptics—Quantitative suspension test for the evaluation of bactericidal activity of chemical disinfectants used in the food, industrial, domestic and institutional areas; (iiii) European Standard Surface Test EN 13697:2001—Chemical disinfectants and antiseptics—Quantitative non-porous surface test for the evaluation of bactericidal and/or fungicidal activity of chemical disinfectants used in food, industrial, domestic and institutional areas; (v) ASTM E1153—Standard Test Method for Efficacy of Sanitizers Recommended for Inanimate Non-Food Contact Surfaces.

10. A treatment composition according to claim 1, wherein the treatment compositions exhibit at least a 3 log₁₀ reduction of one or more undesired microorganisms (pathogens), of at least 3.25 of at least one, preferably at least two or more of: Salmonella enterica, Staphylococcus aureus, Pseudomonas aeruginosa, Escherichia coli, Enterococcus hirae, Candida albicans and Aspergillis niger as demonstrated according to one or more of the follow known test protocols: (i) AOAC Official Method 961.02 Germicidal Spray Products as Disinfectants, including also when modified for use with towelettes according to EPA Series 810 guidelines (OCSPP 810.2200: Disinfectants for Use on Hard Surfaces—Efficacy Data Recommendations), (ii) AOAC Official Method 955.14, 955.15 (preferably against S. aureus and/or S. enterica); (iii) European Standard EN 1276:2009-Chemical Disinfectants and Antiseptics—Quantitative suspension test for the evaluation of bactericidal activity of chemical disinfectants used in the food, industrial, domestic and institutional areas; (iiii) European Standard Surface Test EN 13697:2001—Chemical disinfectants and antiseptics—Quantitative non-porous surface test for the evaluation of bactericidal and/or fungicidal activity of chemical disinfectants used in food, industrial, domestic and institutional areas; (v) ASTM E1153—Standard Test Method for Efficacy of Sanitizers Recommended for Inanimate Non-Food Contact Surfaces.

11. A carrier substrate which contains a quantity of a hard surface treatment composition according to claim 1.

12. A method of treating an animate and/or inanimate surface upon which is known to be present or suspected to be present one or more undesired microorganisms, preferably one or more of bacteria selected from the genus: Salmonella, especially S. enterica, and/or of the genus Staphylococcus, especially Staphylococcus aureus and/or of the genus Pseudomonas, especially P. aeruginosa, and/or of the genus Eserichia, especially E. coli, and/or of the genus Enterococcus, and especially E. hirae, and/or further microorganisms (yeasts, fungi) of the genus Candida, preferably C. albicans and/or of the genus Aspergillis, preferably A. niger, which method includes the step of: applying, (optionally repeatably applying,) an antimicrobially effective amount of a treatment composition according to claim 1 to the said surface in order to reduce the incidence of the aforesaid microorganisms (pathogens) associated with and/or upon the said surface.

13. The treatment composition of claim 1, wherein the (c) an anionic hydrotrope surfactant compound(s) includes a cumene sulfonate compound.

14. The treatment composition of claim 2, wherein the (c) an anionic hydrotrope surfactant compound(s) includes a xylene sulfonate compound and/or a cumene sulfonate compound.

15. The treatment composition of claim 3, wherein the (c) aromatic hydrotrope compound(s) includes a cumene sulfonate compound.

16. The treatment composition of claim 4, wherein the (c) aromatic hydrotrope compound(s) includes a cumene sulfonate compound.

17. The treatment composition of claim 5, wherein the (b) an anionic aromatic hydrotrope compound is selected from the group consisting of: cumene sulfonate and/or xylene sulfonate or salts thereof.

18. The treatment composition of claim 5, wherein the treatment composition exhibits a pH of from about 1.8 to about 3.05.

19. The treatment composition of claim 6, wherein the (b) one or more anionic aromatic hydrotrope compounds is selected from the group consisting of: cumene sulfonate and/or xylene sulfonate or salts thereof.

20. The treatment composition of claim 6, wherein the (b) one or more anionic surfactants include both linear alkane sulfonate and a linear alkane sulfate.

21. The treatment composition of claim 6, wherein the composition has a pH of about 2 to about 3.05.

22. The treatment composition of claim 7, wherein the (b) one or more anionic aromatic hydrotrope compounds is selected from the group consisting of: cumene sulfonate and/or xylene sulfonate or salts thereof.

23. The treatment composition of claim 7, wherein the (b) one or more anionic surfactants include both linear alkane sulfonate and a linear alkane sulfate.

24. The treatment composition of claim 7, wherein the composition has a pH of about 2 to about 3.05.
